(19) United States
(12) Patent Application Publication
(10) Pub. No.: US 2026/0094233 A1
(43) Pub. Date: Apr. 2, 2026

(54) X-RAY IMAGING APPARATUS

(71) Applicant: SHIMADZU CORPORATION, Kyoto-shi (JP)

(72) Inventors: Yuki HARADA, Kyoto-shi (JP); Fumihiro SOMA, Kyoto-shi (JP); Naohiko WAKAYAMA, Kyoto-shi (JP)

(21) Appl. No.: 19/328,885

(22) Filed: Sep. 15, 2025

(30) Foreign Application Priority Data
Sep. 27, 2024 (JP) 2024-168899

(51) Int. Cl. G06T 3/40; A61B 6/00; A61B 6/46

(52) U.S. Cl. CPC G06T 3/40; A61B 6/461; A61B 6/467; A61B 6/54

(57) ABSTRACT

An X-ray imaging apparatus 100 comprises an X-ray irradiation unit 1, an X-ray detection unit 2 that outputs an image signal, a collimator 3, a control unit 6, and an image generation unit 4. The control unit 6 switches the output mode of the X-ray detection unit 2 to a first mode, in which the time required for outputting the image signal is relatively long but the visibility of the generated X-ray image 40 is relatively high, when the entire X-ray irradiation range 50 is included in a first region 20a. It switches the output mode of the X-ray detection unit 2 to a second mode, in which the time required for outputting the image signal is relatively small but the visibility of the generated X-ray image 40 is relatively low, when at least a part of the X-ray irradiation range 50 is included in a second region 20b located outside the first region 20a.

### TECHNICAL FIELD

The present invention relates to an X-ray imaging apparatus, and more particularly, to an X-ray imaging apparatus that performs imaging by adjusting an X-ray irradiation range in accordance with the movement of a region of interest in an X-ray image.

### BACKGROUND ART

Conventionally, an X-ray imaging apparatus is known that performs imaging by adjusting an X-ray irradiation range in accordance with the movement of a region of interest in an X-ray image (see, for example, Patent Literature 1).

Patent Literature 1 discloses an X-ray diagnostic apparatus (X-ray imaging apparatus) that includes a top plate on which a subject is placed, an X-ray tube that irradiates X-rays, an X-ray detector that detects X-rays transmitted through the subject, and a collimator device that forms an X-ray irradiation range on the X-ray detector. The X-ray diagnostic apparatus disclosed in Patent Literature 1 discloses a configuration in which, when the examination target of the subject placed on the top plate is changed, and if the changed examination target is within the detection range of the X-ray detector, the collimator device is operated to move the center of the irradiation field. The configuration disclosed in Patent Literature 1 discloses a configuration in which the collimator device is operated to move the center of the irradiation field to align the center position of the irradiation field with the changed examination target. Furthermore, in the configuration disclosed in Patent Literature 1, moving the top plate when an endoscope is inserted into the subject's body would place a significant burden on the subject, so the center of the irradiation field is moved by operating the collimator device without moving the top plate.

### CITATION LIST

### PATENT LITERATURE

[Patent Literature 1] Japanese Unexamined Patent Application Publication No. 2009-077759

### SUMMARY OF INVENTION

### TECHNICAL PROBLEM

Although not explicitly stated in Patent Literature 1, when generating an X-ray image, it may be difficult to generate an X-ray image with a predetermined visibility depending on the positional relationship between the irradiation field (X-ray irradiation range) and the X-ray detector (X-ray detection unit). Specifically, an X-ray image is generated based on an image signal output from the X-ray detection unit. The X-ray detection unit can detect X-rays only in a central area (first region). In the first region, although the number of pixels is smaller than in the entire area of the X-ray detection unit, and thus the time required for outputting the image signal is relatively long, it is possible to generate an X-ray image with relatively high visibility. However, in a region other than the first region (second region), due to the constraints of the X-ray detection unit, it is necessary to generate an X-ray image based on the image signal output from the entire area of the X-ray detection unit. Therefore, it becomes difficult to generate an X-ray image with visibility equivalent to that when generating an X-ray image based on the image signal in the first region. As a countermeasure, it is conceivable to lower the visibility when generating an X-ray image based on the image signal in the first region to match the visibility of the X-ray image based on the image signal in the second region. However, in this case, the visibility of the X-ray image based on the image signal in the central area is intentionally degraded, which is not preferable.

Therefore, it is conceivable to change the output mode of the image signal in the X-ray detection unit between the first region and the second region. In this case, an operator may set the output mode of the image signal in the X-ray detector to change the visibility of the X-ray image. However, when the operator sets the output mode of the image signal in the X-ray detector to change the visibility of the X-ray image, the operation becomes complicated, and the observation of the subject is temporarily interrupted, which leads to a problem of decreased operator convenience (usability).

The present invention has been made to solve the above problems, and an object of the present invention is to provide an X-ray imaging apparatus capable of suppressing complicated operations and temporary interruption of subject observation, which are caused by the operation of changing the output mode of the image signal in the X-ray detection unit accompanying the movement of the X-ray irradiation range.

### SOLUTION TO PROBLEM

An X-ray imaging apparatus according to a first aspect of the present invention comprises: an X-ray irradiation unit that irradiates X-rays; an X-ray detection unit that has an opposing surface facing the X-ray irradiation unit and outputs an image signal based on the X-rays irradiated by the X-ray irradiation unit; an irradiation range changing unit that changes the position of the irradiation range of the X-rays irradiated from the X-ray irradiation unit on the opposing surface; an image generation unit that generates an X-ray image based on the image signal output by the X-ray detection unit; a display unit that displays the X-ray image generated by the image generation unit; and a control unit, wherein the X-ray detection unit has a first region, which is a predetermined region on the opposing surface, and is capable of switching an output mode between a first mode, in which the time required for outputting the image signal in a predetermined range of the opposing surface is relatively long but the visibility of the X-ray image generated by the image generation unit is relatively high, and a second mode, in which the time required for outputting the image signal in the predetermined range of the opposing surface is relatively small but the visibility of the X-ray image generated by the image generation unit is relatively low, and the control unit determines, as a result of the change in the position of the X-ray irradiation range on the opposing surface by the irradiation range changing unit, whether it is in a first state where the entire X-ray irradiation range is included in the first region, or a second state where at least a part of the X-ray irradiation range is located within a second region outside the first region in the opposing surface, and (a) if it is determined to be in the first state, switches the output mode of the X-ray detection unit to the first mode, and (b) if it is determined to be in the second state, switches the output mode of the X-ray detection unit to the second mode.

An X-ray imaging apparatus according to a second aspect of the present invention comprises: an X-ray irradiation unit that irradiates X-rays; an X-ray detection unit that has an opposing surface facing the X-ray irradiation unit and outputs an image signal based on the X-rays irradiated by the X-ray irradiation unit; an irradiation range changing unit that changes the position of the irradiation range of the X-rays irradiated from the X-ray irradiation unit on the opposing surface; an image generation unit that generates an X-ray image based on the image signal output by the X-ray detection unit; a display unit that displays the X-ray image generated by the image generation unit; and a control unit, wherein the X-ray detection unit has a first region, which is a predetermined region on the opposing surface, and is capable of switching an output mode between a first mode, in which the time required for outputting the image signal in a predetermined range of the opposing surface is relatively long but the visibility of the X-ray image generated by the image generation unit is relatively high, and a second mode, in which the time required for outputting the image signal in the predetermined range of the opposing surface is relatively small but the visibility of the X-ray image generated by the image generation unit is relatively low, and the control unit determines, as a result of the change of the X-ray irradiation range by the irradiation range changing unit, whether it is in a first state where the entire X-ray irradiation range is included in the first region, or a second state where at least a part of the X-ray irradiation range is located within a second region outside the first region in the opposing surface, and if it is determined to be in the second state, deforms the X-ray irradiation range to maintain the first state and sets the output mode of the image signal in the X-ray detection unit to the first mode.

### ADVANTAGEOUS EFFECTS OF INVENTION

In the X-ray imaging apparatus according to the first aspect, the control unit determines, as a result of a change in the position of the X-ray irradiation range on the opposing surface by the irradiation range changing unit, whether it is in a first state where the entire X-ray irradiation range is included in the first region, or a second state where at least a part of the X-ray irradiation range is located within a second region outside the first region in the opposing surface, and if it is determined to be in the first state, switches the output mode of the X-ray detection unit to the first mode, and if it is determined to be in the second state, switches the output mode of the X-ray detection unit to the second mode. As a result, the first mode in which an X-ray image with relatively high visibility is generated and the second mode in which an X-ray image with relatively low visibility is generated are automatically switched, so that the image signal output mode suitable for the positional relationship between the first and second regions and the X-ray irradiation range can be set without the operator changing the image signal output mode of the X-ray detection unit. Consequently, it is possible to suppress complicated operations and temporary interruption of subject observation, which are caused by the operation of changing the output mode of the image signal in the X-ray detection unit accompanying the movement of the X-ray irradiation range.

In the X-ray imaging apparatus according to the second aspect, the control unit determines, as a result of the change in the position of the X-ray irradiation range on the opposing surface by the irradiation range changing unit, whether it is in a first state where the entire X-ray irradiation range is included in the first region, or a second state where at least a part of the X-ray irradiation range is located within a second region outside the first region in the opposing surface, and if it is determined to be in the first state, deforms the X-ray irradiation range to maintain the first state and sets the output mode of the image signal in the X-ray detection unit to the first mode. Therefore, the output mode of the image signal from the X-ray detection unit is not switched from the first mode, in which an X-ray image with relatively high visibility is generated, due to the movement of the X-ray irradiation range, thus suppressing complicated operations and temporary interruption of subject observation caused by changing the image signal output mode to the second mode accompanying the movement of the X-ray irradiation range. Some operators may not require an X-ray image of parts of the subject other than the part they want to observe. Therefore, some operators may prefer the image signal output mode to be set to the first mode, which generates an X-ray image with relatively high visibility, rather than being changed to the second mode with relatively low visibility when the X-ray irradiation range moves to a position straddling the first and second regions. Thus, in the X-ray imaging apparatus according to the second aspect, even when the control unit determines that it is in the second state where at least a part of the X-ray irradiation range is included in the second region as a result of controlling the irradiation range changing unit, it deforms the X-ray irradiation range to maintain the first state and sets the output mode of the image signal in the X-ray detection unit to the first mode. As a result, even when it is determined that it is in the second state where at least a part of the X-ray irradiation range is included in the second region, the first mode, which generates an X-ray image with relatively high visibility, can be maintained. Consequently, it is possible to provide an X-ray imaging apparatus that can meet the needs of operators who wish for the setting to be the first mode with relatively high visibility of the generated X-ray image, even when the X-ray irradiation range moves to a position straddling the first and second regions.

### BRIEF DESCRIPTION OF DRAWINGS

FIG. 1 is a schematic diagram showing the overall configuration of an X-ray imaging apparatus according to a first embodiment.

FIG. 2 is a functional block diagram of the X-ray imaging apparatus according to the first embodiment.

FIG. 3 is a schematic diagram for explaining an X-ray image.

FIG. 4 is a schematic diagram for explaining a first region and a second region in an X-ray detection unit according to the first embodiment.

FIG. 5 is a schematic diagram for explaining the movement of the X-ray irradiation range on an opposing surface.

FIG. 6 is a schematic diagram for explaining an image signal output in a first mode.

FIG. 7 is a schematic diagram for explaining an image signal output in a second mode.

FIG. 8(A) and FIG. 8(B) are schematic diagrams for explaining a configuration for adjusting the X-ray irradiation range by an irradiation range changing unit.

FIG. 9(A) is a schematic diagram for explaining a configuration for generating an X-ray image at a low frame rate, and FIG. 9(B) is a schematic diagram for explaining a configuration for generating an X-ray image at a high frame rate.

FIG. 10(A) to FIG. 10(C) are schematic diagrams for explaining differences in binning size in the X-ray detection unit.

FIG. 11 is a schematic diagram for explaining a configuration for changing the image signal output mode in the first mode and the second mode, and the image signal output mode to be prioritized in the second mode.

FIG. 12 is a flowchart for explaining a process in which a control unit according to the first embodiment changes the image signal output mode.

FIG. 13 is a functional block diagram of an X-ray imaging apparatus according to a second embodiment.

FIG. 14 is a schematic diagram for explaining a configuration in which a control unit according to the second embodiment sets an irradiable region.

FIG. 15 is a schematic diagram for explaining a configuration in which the control unit according to the second embodiment deforms the irradiable range.

FIG. 16 is a schematic diagram for explaining a configuration in which the control unit according to the second embodiment deforms and displays a frame line to match the deformed X-ray irradiation range.

FIG. 17 is a schematic diagram of an X-ray image generated by an image generation unit according to the second embodiment.

FIG. 18 is a flowchart for explaining a process in which the control unit according to the second embodiment deforms the X-ray irradiation range.

### DESCRIPTION OF EMBODIMENTS

Hereinafter, embodiments embodying the present invention will be described based on the drawings.

### First Embodiment

### Configuration of X-ray Imaging Apparatus

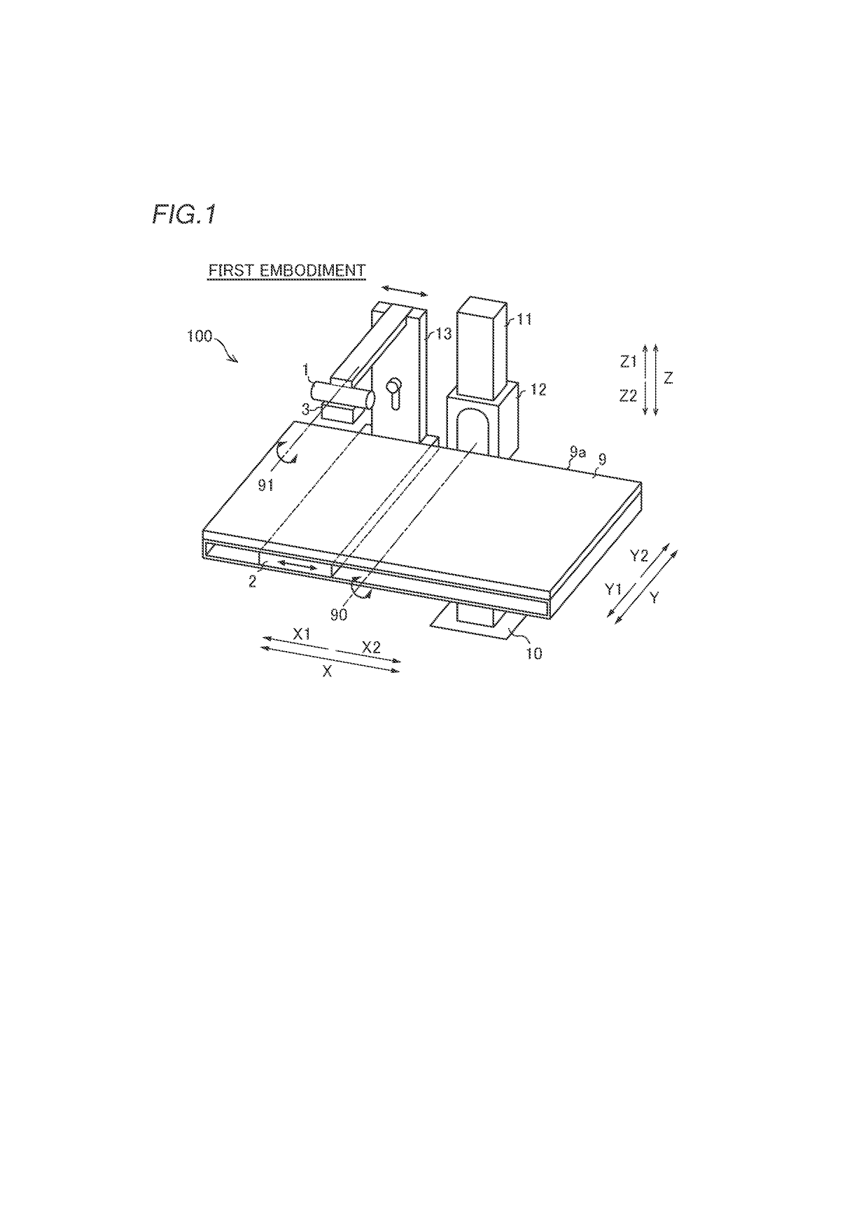

With reference to FIG. 1 to FIG. 12, the configuration of an X-ray imaging apparatus 100 according to the present embodiment will be described.

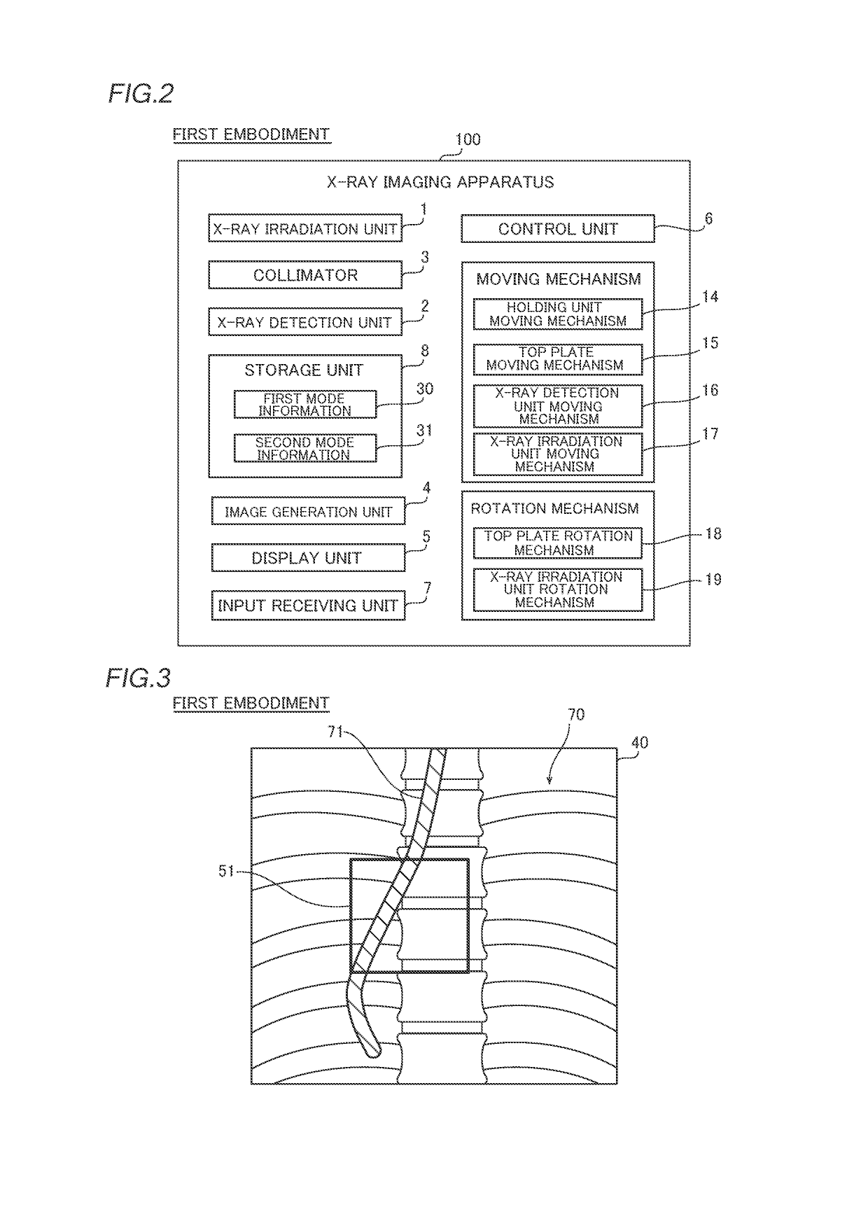

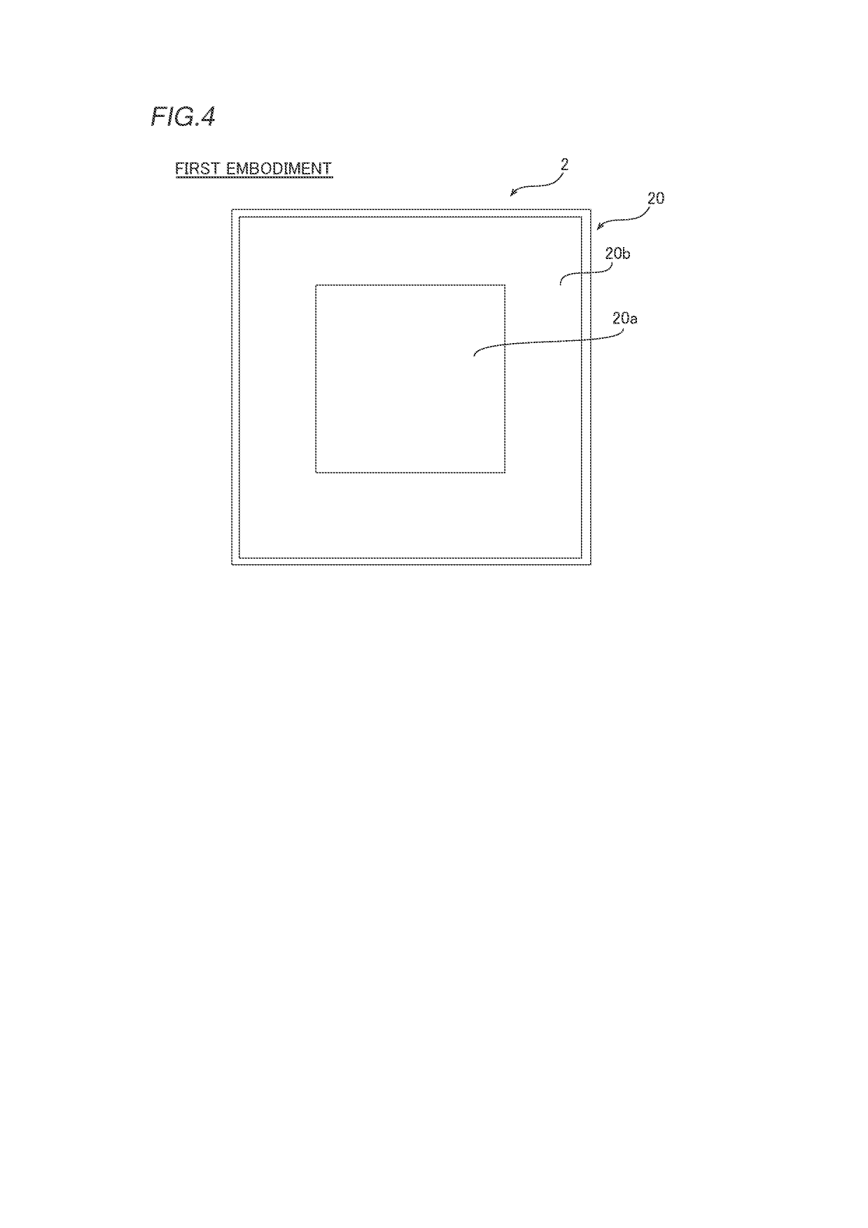

First, with reference to FIG. 1 and FIG. 2, the overall configuration of the X-ray imaging apparatus 100 will be described. As shown in FIG. 1 and FIG. 2, the X-ray imaging apparatus 100 includes an X-ray irradiation unit 1, an X-ray detection unit 2, a collimator 3, an image generation unit 4, a display unit 5, a control unit 6, an input receiving unit 7, a storage unit 8, and a top plate 9. The X-ray imaging apparatus 100 is configured to irradiate a subject with X-rays to acquire an X-ray image 40 (see FIG. 3). The collimator 3 is an example of the "irradiation range changing unit" in the claims.

The X-ray irradiation unit 1 and the X-ray detection unit 2 are used for fluoroscopic imaging of an imaging region of a subject placed on the top plate 9. Here, fluoroscopic imaging refers to a method of acquiring a moving image of an imaging region while irradiating with X-rays at a lower dose than for still image photography.

The X-ray irradiation unit 1 is configured to irradiate X-rays. The X-ray irradiation unit 1 includes an X-ray tube that irradiates X-rays by being supplied with power from a power supply device (not shown).

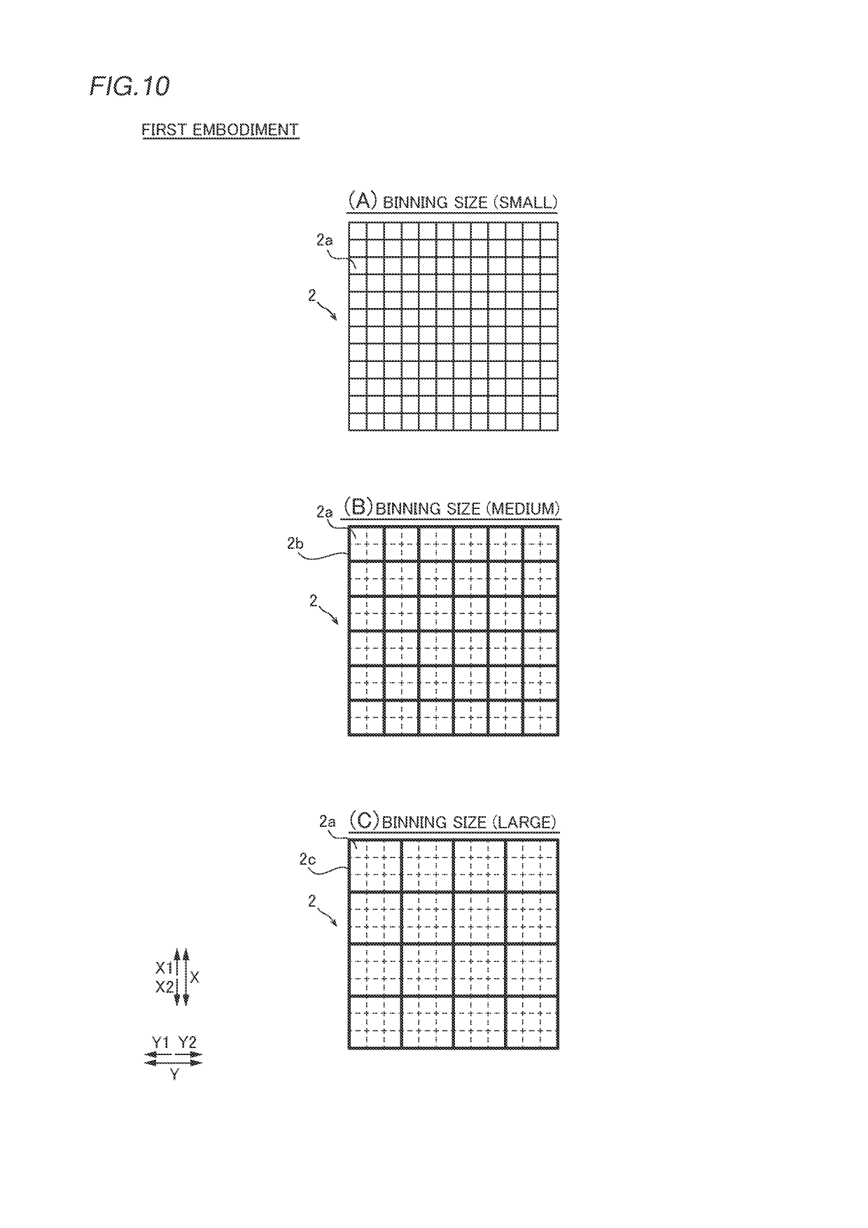

The X-ray detection unit 2 has an opposing surface 20 (see FIG. 4) facing the X-ray irradiation unit 1. The X-ray detection unit 2 has a plurality of pixels 2a (see FIG. 10(A)) provided below the opposing surface 20 for accumulating electric charge. The X-ray detection unit 2 is, for example, an FPD (Flat Panel Detector). The X-ray detection unit 2 is configured to output an image signal based on the X-rays irradiated by the X-ray irradiation unit 1.

In the X-ray imaging apparatus 100, the X-ray irradiation unit 1 is provided on the front surface side of the top plate 9, and the X-ray detection unit 2 is provided on the back surface side of the top plate 9. The X-ray irradiation unit 1 and the X-ray detection unit 2 are provided to face each other with the top plate 9 interposed therebetween.

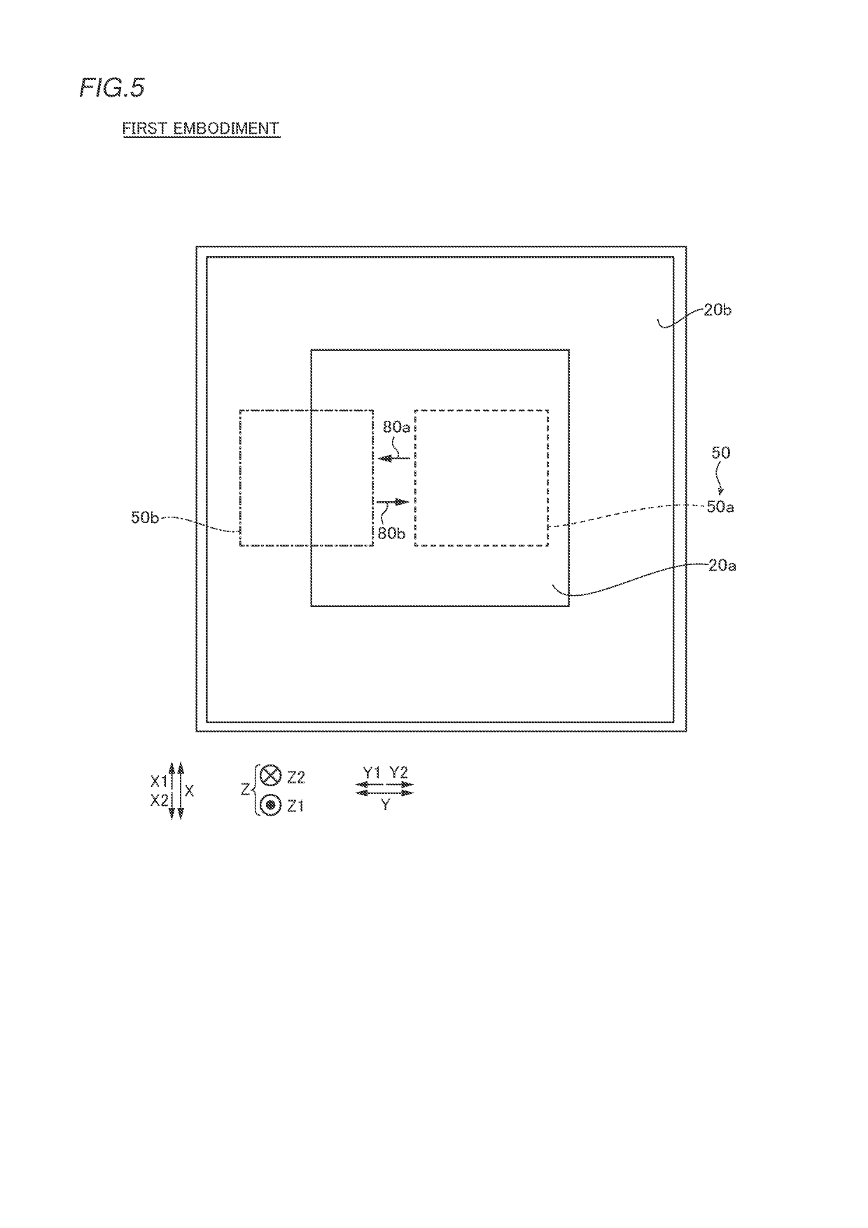

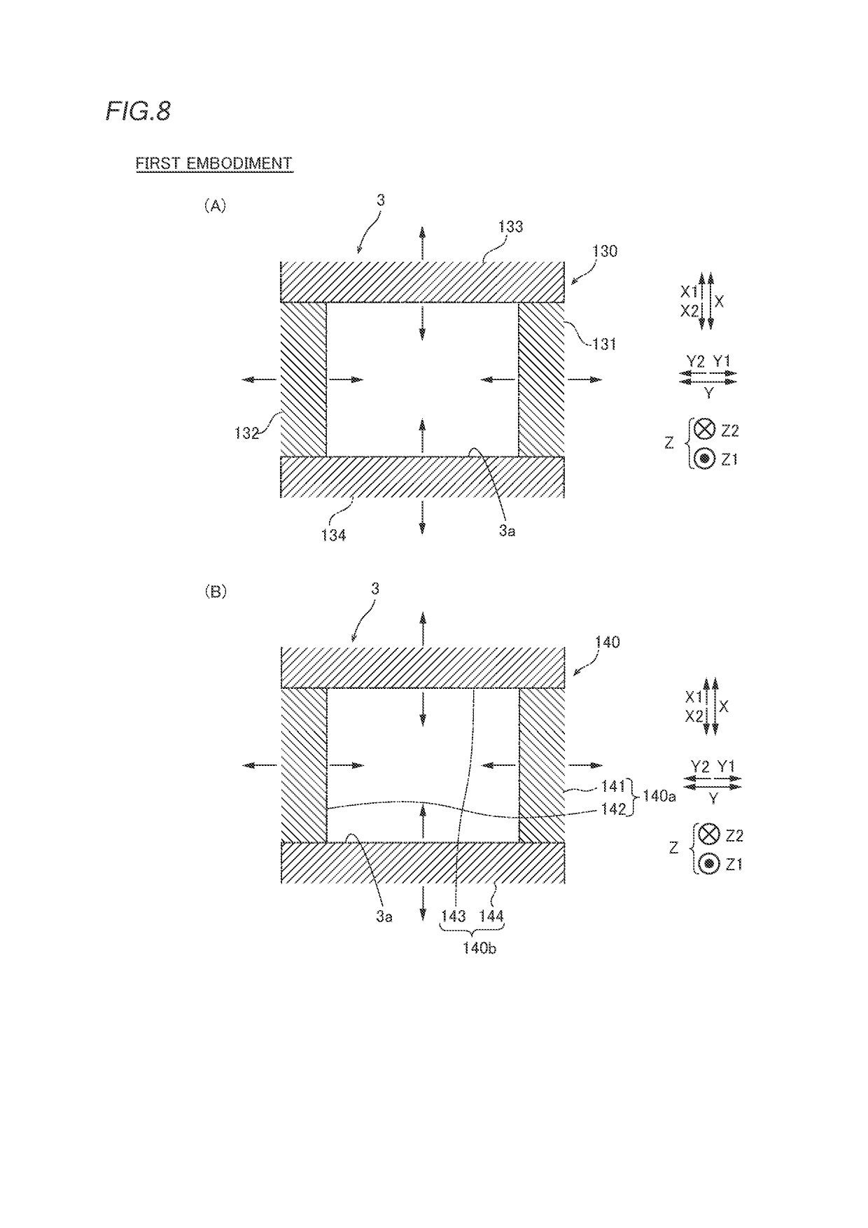

The collimator 3 is configured to change the position of the irradiation range 50 (see FIG. 5) of the X-rays irradiated from the X-ray irradiation unit 1 on the opposing surface 20. The collimator 3 is disposed in front of the X-ray emission direction. Inside the collimator 3, a first group of a plurality of shielding blades 130 provided on the X-ray tube side, as shown in FIG. 8(A), and a second group of a plurality of shielding blades 140 provided on the top plate 9 side, as shown in FIG. 8(B), are provided. The X-rays irradiated from the X-ray tube pass through an opening 3a (X-ray irradiation range 50) formed by the first group of the plurality of shielding blades 130 and the second group of the plurality of shielding blades 140. The details of the configuration in which the collimator 3 adjusts the opening 3a (X-ray irradiation range 50) by the first group of the plurality of shielding blades 130 and the second group of the plurality of shielding blades 140 will be described later.

The image generation unit 4 is configured to generate an X-ray image 40 based on the image signal output by the X-ray detection unit 2. In the present embodiment, the image generation unit 4 generates the X-ray image 40 as a moving image. The image generation unit 4 includes a GPU (Graphics Processing Unit) or an FPGA (Field-Programmable Gate Array) configured for image processing.

The display unit 5 is configured to display a plurality of X-ray images 40 generated by the image generation unit 4. The display unit 5 includes a display device such as a liquid crystal monitor or an organic EL (Electro Luminescence) monitor.

The control unit 6 is configured to control the collimator 3 and the output mode of the image signal in the X-ray detection unit 2. The control unit 6 includes a processor or circuitry such as a CPU (Central Processing Unit), and a memory such as a ROM (Read Only Memory) and a RAM (Random Access Memory).

The input receiving unit 7 is configured to receive an operation input from an operator. The input receiving unit 7 includes, for example, a keyboard and a pointing device such as a mouse. The display unit 5 and the input receiving unit 7 may be integrally formed as a touch panel.

The storage unit 8 stores various programs executed by the control unit 6. The storage unit 8 also stores first mode information 30 and second mode information 31. The first mode information 30 and the second mode information 31 are conditions for the mode of outputting the image signal output by the X-ray detection unit 2. The details of the conditions for the mode of outputting the image signal by the X-ray detection unit 2 will be described later.

The top plate 9 is configured to place a subject on it. The top plate 9 has a placement surface 9a for placing the subject. In this description, the X direction is the longitudinal direction of the top plate 9 when the top plate 9 is in a horizontal state. The Y direction is the short-side direction of the top plate 9 when the top plate 9 is in a horizontal state. The Z direction is the vertical direction.

The X-ray imaging apparatus 100 includes, as a support mechanism, a base 10, a first support column 11, a holding unit 12, and a second support column 13. As shown in FIG. 2, the X-ray imaging apparatus 100 also includes, as a moving mechanism, a holding unit moving mechanism 14, a top plate moving mechanism 15, an X-ray detection unit moving mechanism 16, and an X-ray irradiation unit moving mechanism 17. The X-ray imaging apparatus 100 also includes, as a rotation mechanism, a top plate rotation mechanism 18 and an X-ray irradiation unit rotation mechanism 19.

As shown in FIG. 1, the first support column 11 supports the entire X-ray imaging apparatus 100. The first support column 11 is provided on the base 10. The first support column 11 is provided with a holding unit moving mechanism 14 (see FIG. 2). The holding unit moving mechanism 14 allows the holding unit 12 to be movable in the Z direction.

The holding unit 12 holds the top plate 9, the second support column 13, and the X-ray detection unit 2. The holding unit 12 is provided with a top plate moving mechanism 15 (see FIG. 2), an X-ray detection unit moving mechanism 16 (see FIG. 2), and a top plate rotation mechanism 18 (see FIG. 4). The top plate moving mechanism 15 allows the top plate 9 to be movable in the short-side direction of the top plate 9 (Y direction in FIG. 1). The X-ray detection unit moving mechanism 16 allows the X-ray detection unit 2 to be movable in the longitudinal direction of the top plate 9 (X direction in FIG. 2). The XY directions in FIG. 2 are substantially horizontal directions. The top plate rotation mechanism 18 allows the top plate 9 to be rotatable around an axis 90 extending along the short-side direction (Y direction) of the top plate 9.

The second support column 13 supports the X-ray irradiation unit 1. The second support column 13 is provided with an X-ray irradiation unit moving mechanism 17 (see FIG. 4) and an X-ray irradiation unit rotation mechanism 19 (see FIG. 2). The X-ray irradiation unit moving mechanism 17 allows the X-ray irradiation unit 1 to be movable in the longitudinal direction of the top plate 9 (X direction in FIG. 2). The X-ray irradiation unit 1 and the X-ray detection unit 2 can move integrally with respect to the top plate 9 by the synchronous operation of the X-ray irradiation unit moving mechanism 17 and the X-ray detection unit moving mechanism 16. The X-ray irradiation unit rotation mechanism 19 allows the X-ray irradiation unit 1 to be rotatable around an axis 91 extending along the short-side direction (Y direction) of the top plate 9.

### X-ray Image

FIG. 3 is a schematic diagram of an X-ray image 40. As shown in FIG. 3, a bone 70, a device 71, and the like are captured in the X-ray image 40. The device 71 includes, for example, a catheter or an endoscope. A region of interest 51 is displayed superimposed on the X-ray image 40. The region of interest 51 is a region that includes a site that the operator wants to check.

In the first embodiment, the control unit 6 (see FIG. 2) is configured to perform control to move the region of interest 51 in the X-ray image 40. Specifically, when an operation input to move the region of interest 51 is input via the input receiving unit 7 (see FIG. 2), the control unit 6 moves the region of interest 51 in response to the operation input. When the region of interest 51 is set at the tip of the device 71 or the like, the control unit 6 may recognize the tip of the device 71 and move the region of interest 51 in accordance with the position of the tip of the device 71.

### First Region and Second Region

FIG. 4 shows the opposing surface 20 of the X-ray detection unit 2. The opposing surface 20 includes at least a first region 20a and a second region 20b. The first region 20a is a predetermined region on the opposing surface 20. The second region 20b is a region on the opposing surface 20 outside the first region 20a.

### Movement of Irradiation Range

In the first embodiment, the control unit 6 (see FIG. 2) is configured to perform control to move the X-ray irradiation range 50 based on the movement of the region of interest 51 (see FIG. 3). For example, as shown in FIG. 5, the control unit 6 moves an X-ray irradiation range 50a, which is located in the first region 20a and adjusted to have a size (area) smaller than the first region 20a, to a position straddling the first region 20a and the second region 20b, as indicated by arrow 80a. Further, the control unit 6 moves an X-ray irradiation range 50b, which is disposed at a position straddling the first region 20a and the second region 20b, into the first region 20a, as indicated by arrow 80b. The control unit 6 controls the collimator 3 (see FIG. 2) to move the X-ray irradiation range 50 without moving the top plate 9 (see FIG. 1).

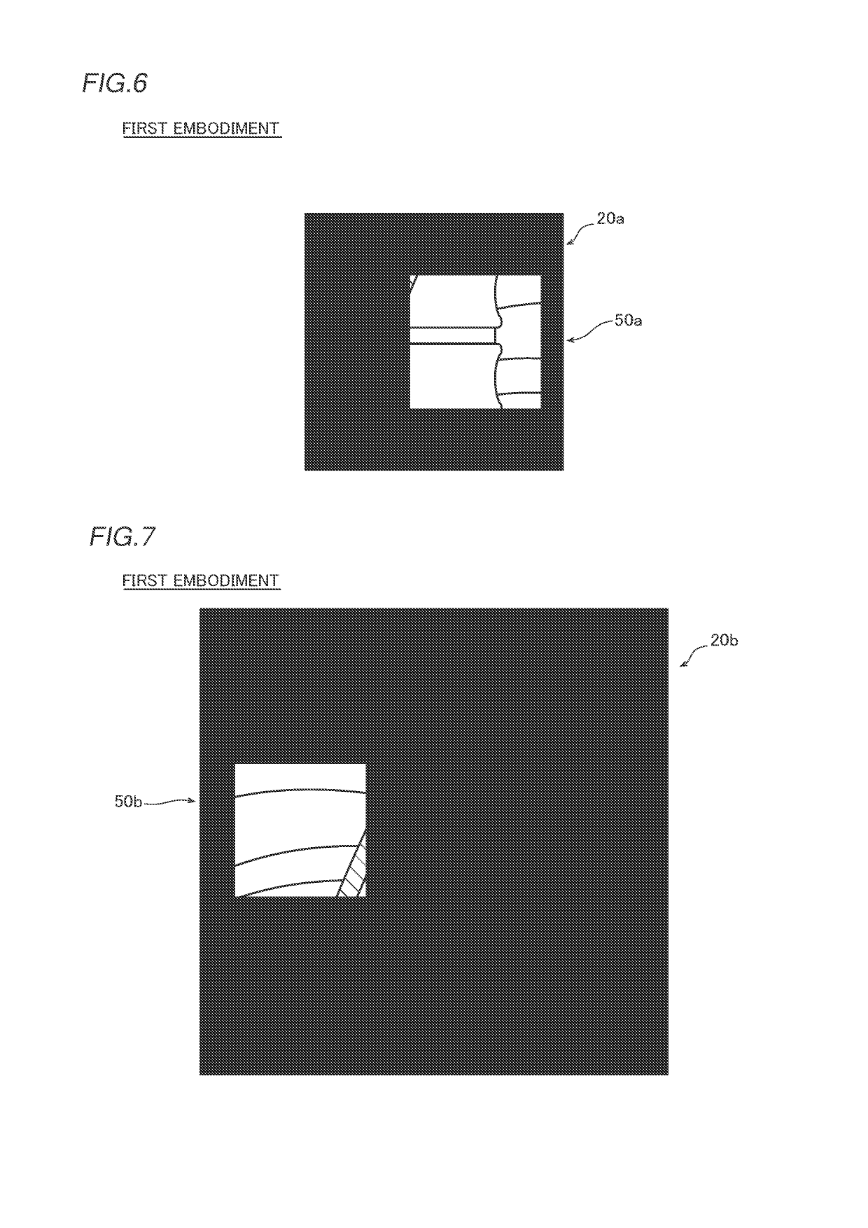

When the X-ray irradiation range 50a is located in the first region 20a, the X-ray detection unit 2 outputs an image signal from the first region 20a. Further, X-rays are irradiated only within the X-ray irradiation range 50a of the first region 20a. Therefore, as shown in FIG. 6, the image signal within the X-ray irradiation range 50a of the first region 20a has pixel values corresponding to the subject's site. Thus, the region within the X-ray irradiation range 50a is imaged. In the region of the first region 20a other than the X-ray irradiation range 50a, no X-rays are irradiated, so there is no image signal. Therefore, it is displayed as a black-filled region in the X-ray image 40.

When the X-ray irradiation range 50a is disposed at a position straddling the first region 20a and the second region 20b, the X-ray detection unit 2 outputs an image signal from the entire opposing surface 20 (the first region 20a and the second region 20b). Further, X-rays are irradiated only within the X-ray irradiation range 50a of the opposing surface 20. Therefore, as shown in FIG. 7, the image signal within the X-ray irradiation range 50a of the opposing surface 20 has pixel values corresponding to the subject's site. Thus, the region within the X-ray irradiation range 50a is imaged. In the region of the opposing surface 20 other than the X-ray irradiation range 50a, no X-rays are irradiated, so there is no image signal. Therefore, it is displayed as a black-filled region in the X-ray image 40.

### Change of X-ray Irradiation Range by Collimator

As shown in FIG. 8(A), the first group of the plurality of shielding blades 130 includes a first shielding blade 131, a second shielding blade 132, a third shielding blade 133, and a fourth shielding blade 134. The first shielding blade 131 is provided on the Y1 side inside the collimator 3 and is configured to be able to narrow the irradiated X-rays by moving in the Y2 direction. The second shielding blade 132 is provided on the Y2 side inside the collimator 3 and is configured to be able to narrow the irradiated X-rays by moving in the Y1 direction. The third shielding blade 133 is provided on the X1 side inside the collimator 3 and is configured to be able to narrow the irradiated X-rays by moving in the X2 direction. The fourth shielding blade 134 is provided on the X2 side inside the collimator 3 and is configured to be able to narrow the irradiated X-rays by moving in the X1 direction.

The first shielding blade 131, the second shielding blade 132, the third shielding blade 133, and the fourth shielding blade 134 are configured to be movable independently of each other. Each of the first shielding blade 131, the second shielding blade 132, the third shielding blade 133, and the fourth shielding blade 134 is configured to be movable in response to an operation input to the input receiving unit 7 (see FIG. 2).

As shown in FIG. 8(B), the second group of the plurality of shielding blades 140 includes a fifth shielding blade 141, a sixth shielding blade 142, a seventh shielding blade 143, and an eighth shielding blade 144. The fifth shielding blade 141 is provided on the Y1 side inside the collimator 3 and is configured to be able to narrow the irradiated X-rays by moving in the Y2 direction. The sixth shielding blade 142 is provided on the Y2 side inside the collimator 3 and is configured to be able to narrow the irradiated X-rays by moving in the Y1 direction. The fifth shielding blade 141 and the sixth shielding blade 142 are configured as a first pair of shielding blades 140a, which are symmetrically controlled to move toward or away from each other along the Y direction.

The seventh shielding blade 143 is provided on the X1 side inside the collimator 3 and is configured to be able to narrow the irradiated X-rays by moving in the X2 direction. The eighth shielding blade 144 is provided on the X2 side inside the collimator 3 and is configured to be able to narrow the irradiated X-rays by moving in the X1 direction. The seventh shielding blade 143 and the eighth shielding blade 144 are configured as a second pair of shielding blades 140b, which are symmetrically controlled to move toward or away from each other along the X direction.

The first pair of shielding blades 140a, consisting of the fifth shielding blade 141 and the sixth shielding blade 142, and the second pair of shielding blades 140b, consisting of the seventh shielding blade 143 and the eighth shielding blade 144, are each configured to be movable in response to an operation input to the input receiving unit 7 (see FIG. 2).

That is, the first group of the plurality of shielding blades 130 is configured to individually adjust the position of each side of the X-ray irradiation range 50. The second group of the plurality of shielding blades 140 is configured to adjust the width (left-right dimension) and the length (up-down dimension) of the X-ray irradiation range 50 without changing the center position of the X-ray irradiation range 50. The control unit 6 sets the X-ray irradiation range 50 based on the set region of interest 51 and irradiates X-rays from the X-ray irradiation unit 1 to capture an X-ray image 40 of the region of interest 51.

Here, the X-ray detection unit 2 outputs an image signal based on the X-rays irradiated by the X-ray irradiation unit 1 onto the opposing surface 20 (see FIG. 4). The image generation unit 4 (see FIG. 2) generates an X-ray image 40 based on the image signal output by the X-ray detection unit 2. In the first embodiment, since the image generation unit 4 generates the X-ray image 40 as a moving image, the visibility of the X-ray image 40 is determined by at least one of the frame rate (number of frames per unit time) and the resolution.

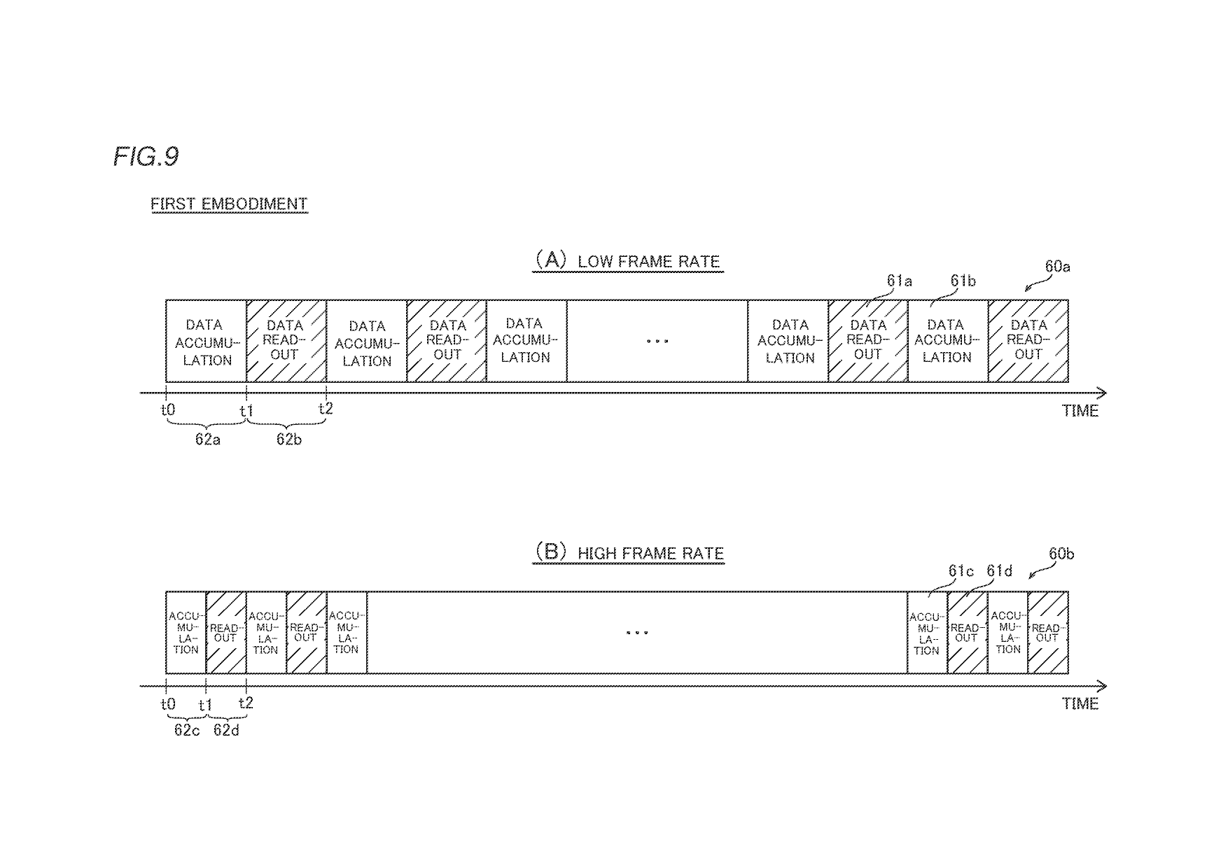

A graph 60a shown in FIG. 9(A) is a graph for a case where the frame rate for generating the X-ray image 40 (see FIG. 3) as a moving image is low. The graph 60a has a horizontal axis representing time. As shown in the graph 60a, when the image generation unit 4 (see FIG. 2) generates the X-ray image 40 as a moving image, it repeats a data accumulation period 61a and a data readout period 61b. The frame rate of the moving image is determined by the sum of one data accumulation period 61a and one data readout period 61b. That is, the frame rate is determined by the sum of the time 62a from time t0 to time t1 and the time 62b from time t1 to time t2. The data accumulation period 61a is a period during which charge is accumulated in the plurality of pixels 2a of the X-ray detection unit 2. The data readout period 61b is a period during which the charge accumulated in the plurality of pixels 2a of the X-ray detection unit 2 is read out.

A graph 60b shown in FIG. 9(B) is a graph for a case where the frame rate for generating the X-ray image 40 (see FIG. 3) as a moving image is high. The graph 60b has a horizontal axis representing time. The data accumulation period 61c and the data readout period 61d in the graph 60b are shorter than the data accumulation period 61a and the data readout period 61b in the graph 60a. That is, in the graph 60b, the sum of the time 62c from time t0 to time t1 and the time 62d from time t1 to time t2 is shorter than the sum of the time 62a from time t0 to time t1 and the time 62b from time t1 to time t2 in the graph 60a. Therefore, the X-ray image 40 generated under the conditions of the data accumulation period 61c and the data readout period 61d shown in the graph 60b has a higher frame rate than the X-ray image 40 generated under the conditions of the data accumulation period 61a and the data readout period 61b shown in the graph 60a. The data accumulation period 61c is a period during which charge is accumulated in the plurality of pixels 2a of the X-ray detection unit 2. The data readout period 61d is a period during which the charge accumulated in the plurality of pixels 2a of the X-ray detection unit 2 is read out.

FIG. 10 shows a configuration for changing the number of pixels from which image signals are output from the X-ray detection unit 2. Specifically, the X-ray detection unit 2 is configured to change the number of pixels from which image signals are output by hardware binning.

FIG. 10 shows examples of three types of binning sizes. FIG. 10(A) shows a case where the binning size is "small," FIG. 10(B) shows a case where the binning size is "medium," and FIG. 10(C) shows a case where the binning size is "large." In FIG. 10(A), image signals are output from each pixel 2a included in the X-ray detection unit 2. In FIG. 10(B), among the pixels 2a included in the X-ray detection unit 2, four pixels 2a in a 2x2 arrangement are regarded as one pixel 2b, and an image signal is output. In FIG. 10(C), among the pixels 2a included in the X-ray detection unit 2, nine pixels 2a in a 3x3 arrangement are regarded as one pixel 2c, and an image signal is output. Therefore, as the binning size increases, the number of pixels outputting image signals decreases, so the time required for outputting the image signals is shortened. However, as the binning size increases, the number of pixels outputting image signals decreases, so the resolution of the X-ray image 40 decreases.

The first region 20a (see FIG. 4) in the central portion of the opposing surface 20 (see FIG. 4) has fewer pixels compared to the entire opposing surface 20. Therefore, even when the time required for image signal output is increased by reducing the binning size to improve the visibility of the X-ray image 40, a high frame rate can be maintained. Thus, when generating the X-ray image 40 (see FIG. 3) as a moving image based on X-rays detected only in the first region 20a, an X-ray image 40 with relatively high visibility (at a predetermined frame rate and a predetermined resolution) can be generated. However, when at least a part of the X-ray irradiation range 50 (see FIG. 5) is located in the second region 20b (see FIG. 4) outside the first region 20a of the opposing surface 20, it is necessary to output image signals from all pixels 2a of the opposing surface 20 due to the equipment constraints of the X-ray detection unit 2. Therefore, when the X-ray irradiation range 50 is located in the second region 20b, the number of pixels 2a outputting image signals increases, which may make it difficult to generate an X-ray image 40 with visibility equivalent to that of the X-ray image 40 generated based on X-rays detected only in the first region 20a.

Therefore, in the first embodiment, the control unit 6 determines, as a result of the change in the position of the X-ray irradiation range 50 on the opposing surface 20 by the collimator 3, whether the apparatus is in a first state where the entire X-ray irradiation range is included in the first region 20a, or in a second state where at least a part of the X-ray irradiation range 50 is included in the second region 20b, which is within the opposing surface 20 but outside the first region 20a. Then, if it is determined to be in the first state, the control unit 6 switches the output mode of the X-ray detection unit 2 to a first mode. If it is determined to be in the second state, the control unit 6 switches the output mode of the X-ray detection unit 2 to a second mode.

In the first embodiment, when outputting an image signal from the X-ray detection unit 2 in the first mode, the control unit 6 generates an X-ray image 40 with relatively high visibility due to a predetermined frame rate and a predetermined resolution. The predetermined frame rate is, for example, 30 fps (frames per second). The predetermined resolution is, for example, a "small" binning size where image signals are output from each pixel 2a of the X-ray detection unit 2 as is, or a "medium" binning size where four 2x2 pixels 2a of the X-ray detection unit 2 are regarded as one pixel 2b for image signal output.

Furthermore, the control unit 6 is configured to lower either the frame rate or the resolution of the X-ray image 40 when making the visibility of the X-ray image 40 generated based on the image signal output in the second mode lower than the visibility of the X-ray image 40 generated based on the image signal output in the first mode. When lowering the frame rate of the X-ray image 40 in the second mode, for example, the control unit 6 sets the frame rate of the generated X-ray image 40 to 15 fps. When lowering the resolution of the plurality of X-ray images 40 generated based on the image signal output in the second mode, the control unit 6 sets a binning size larger than the binning size in the first mode. For example, if the binning size in the first mode is "small," the control unit 6 sets the binning size to "medium" in the second mode. If the binning size in the first mode is "medium," the control unit 6 sets the binning size to "large" in the second mode, where nine 3x3 pixels 2a of the X-ray detection unit 2 are regarded as one pixel 2c for image signal output.

In the first embodiment, the control unit 6 stores the frame rate and resolution of the X-ray image 40 generated based on the image signal output in the first mode in the storage unit 8 (see FIG. 2) as first mode information 30. The control unit 6 also stores the frame rate and resolution of the X-ray image 40 generated based on the image signal output in the second mode in the storage unit 8 as second mode information 31 (see FIG. 2).

In the first embodiment, when the image generation unit 4 generates the X-ray image 40 based on the image signal output by the first mode, it generates an X-ray image 40 with a higher frame rate and resolution than the X-ray image 40 generated based on the image signal output by the second mode. That is, the X-ray image 40 generated based on the image signal output by the first mode is an image with relatively higher visibility than the X-ray image 40 generated based on the image signal output by the second mode. In the first embodiment, the control unit 6 causes the display unit 5 to display the generated X-ray image 40.

### Setting of Readout Conditions for Charge Accumulated in a Plurality of Pixels of X-ray Detection Unit

Here, if the operator has to manually switch the output mode of the image signal from the X-ray detection unit 2 between the first mode and the second mode based on their input, the operation becomes cumbersome. Furthermore, if the mode switching operation is performed during an examination using the device 71 (see FIG. 3), the operator has to look away from the device 71, leading to a temporary interruption of the examination.

Therefore, in the first embodiment, the control unit 6 determines, as a result of the change in the position of the X-ray irradiation range 50 on the opposing surface 20 by the collimator 3, whether the apparatus is in a first state where the entire X-ray irradiation range 50 is included in the first region 20a, or in a second state where at least a part of the X-ray irradiation range 50 is included in the second region 20b, which is within the opposing surface 20 but outside the first region 20a. Then, if it is determined to be in the first state, the control unit 6 switches the output mode of the X-ray detection unit 2 to the first mode. If it is determined to be in the second state, the control unit 6 switches the output mode of the X-ray detection unit 2 to the second mode.

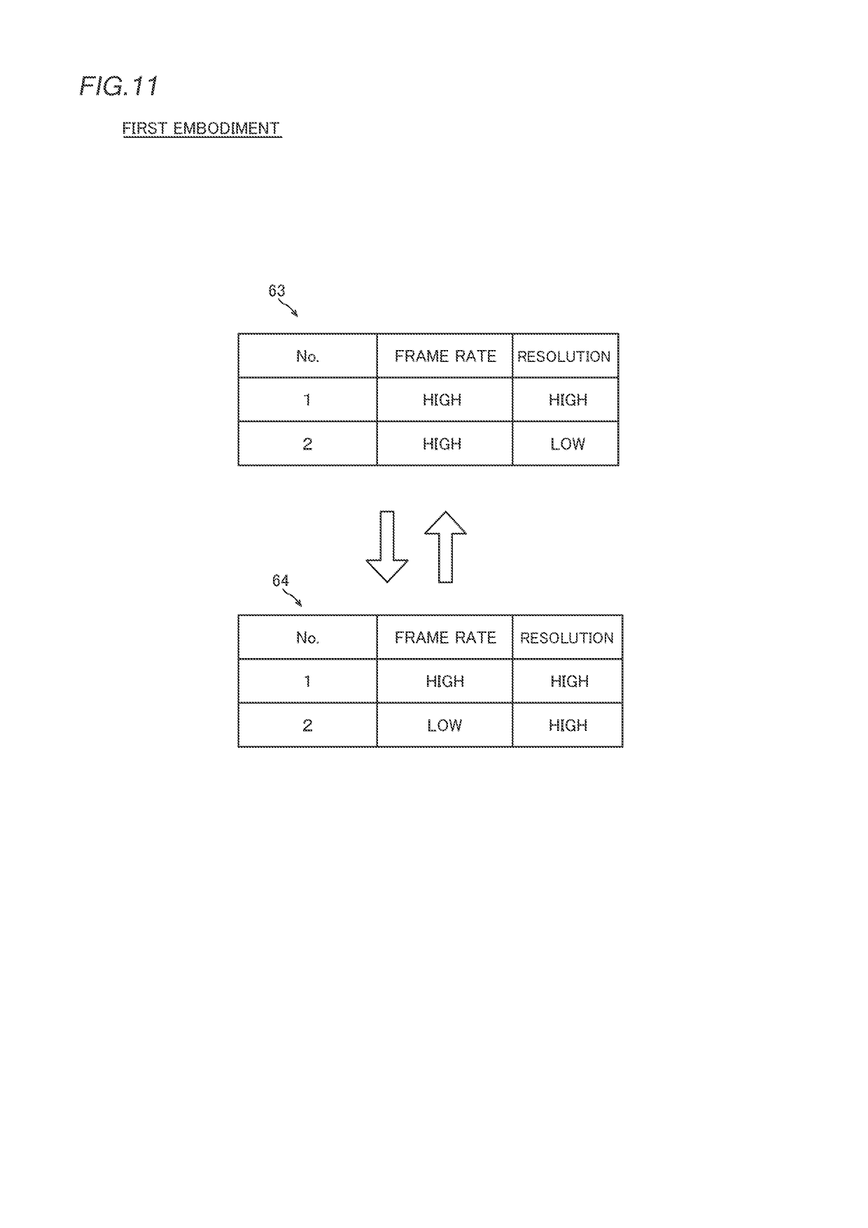

When generating the X-ray image 40 based on the image signal output in the second mode, whether to lower the frame rate or the resolution depends on the operator's preference. That is, some operators prefer to maintain the frame rate even if the resolution decreases, while others prefer to maintain the resolution even if the frame rate decreases. Therefore, in the first embodiment, as shown in FIG. 11, the control unit 6 (see FIG. 2) is configured to allow the operator to set, via an operation input through the input receiving unit 7 (see FIG. 2), whether to lower the frame rate or the resolution of the X-ray image 40 when making the visibility of the X-ray image 40 (see FIG. 3) generated based on the image signal output in the second mode lower than the visibility of the X-ray image 40 generated based on the image signal output in the first mode.

A table 63 shown in FIG. 11 indicates the conditions for lowering the resolution when making the visibility of the X-ray image 40 generated based on the image signal output in the second mode lower than the visibility of the X-ray image 40 generated based on the image signal output in the first mode. A table 64 indicates the conditions for lowering the frame rate when making the visibility of the X-ray image 40 generated based on the image signal output in the second mode lower than the visibility of the X-ray image 40 generated based on the image signal output in the first mode. As shown in tables 63 and 64, the X-ray image 40 generated in the first mode has relatively higher visibility than in the second mode by having both a higher frame rate and higher resolution. Note that a high frame rate means that the frame rate is high compared to the X-ray image 40 generated based on the image signal output in the second mode. For example, if the frame rate of the plurality of X-ray images 40 generated based on the image signal output in the second mode shown in table 64 is 7.5 fps, even if the frame rate of the X-ray image 40 generated based on the image signal output in the first mode is 15 fps, the frame rate is considered high. Similarly, for the resolution shown in table 63, if the resolution of the X-ray image 40 generated based on the image signal output in the first mode is higher than the resolution of the X-ray image 40 generated based on the image signal output in the second mode, it is considered high regardless of the numerical value of the resolution.

The control unit 6 updates the second mode information 31 stored in the storage unit 8 with the image signal output conditions in the second mode set based on the operation input. That is, the control unit 6 overwrites the second mode information 31 that was stored in the storage unit 8 with the frame rate and resolution in the second mode set based on the operation input.

When storing the first mode information 30 and the second mode information 31, the number (No.), frame rate, and resolution are stored in association with each other. Therefore, when switching between the first mode and the second mode, the control unit 6 switches the output mode based on the number. The control unit 6 may also switch between the first mode and the second mode by setting each of the frame rate and resolution, instead of using the number.

### Image Signal Output Mode Change Process

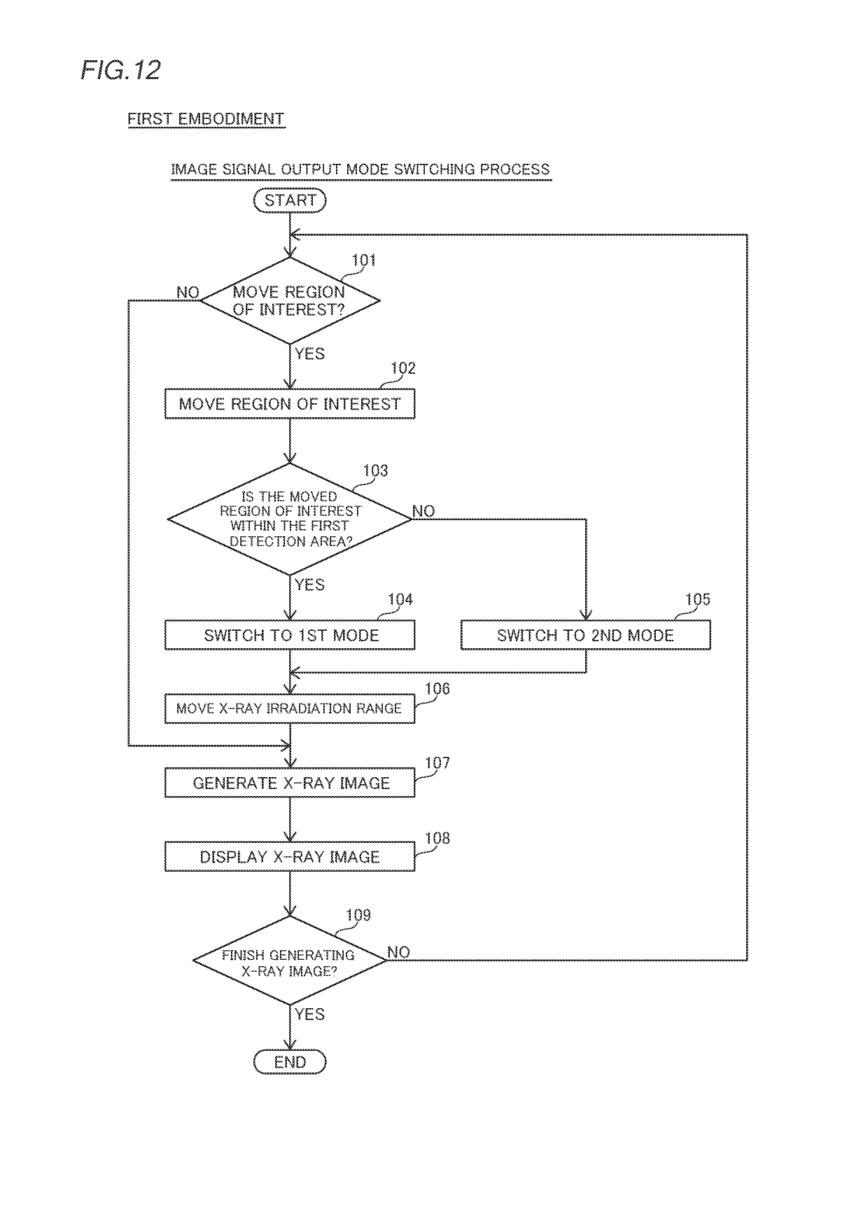

Next, with reference to FIG. 12, a process in which the control unit 6 (see FIG. 2) changes the mode for outputting an image signal from the X-ray detection unit 2 (see FIG. 2) will be described.

In step 101, the control unit 6 determines whether to move the region of interest 51 (see FIG. 3). If the region of interest 51 is to be moved, the process proceeds to step 102. If the region of interest 51 is not to be moved, the process proceeds to step 107.

When the process proceeds from step 101 to step 102, in step 102, the control unit 6 moves the region of interest 51.

Next, in step 103, the control unit 6 determines whether or not the entirety of the moved region of interest 51 is located within the first region 20a (see FIG. 4) of the opposing surface 20 (see FIG. 4) of the X-ray detection unit 2 (see FIG. 4). If the moved region of interest 51 is located within the first region 20a, the process proceeds to step 104. If the moved region of interest 51 is not located within the first region 20a, the process proceeds to step 105.

When the process proceeds from step 103 to step 104, in step 104, the control unit 6 switches the mode for outputting the image signal from the X-ray detection unit 2 to the first mode. If the image signal output mode is already set to the first mode, the process of step 104 is skipped.

When the process proceeds from step 103 to step 105, in step 105, the control unit 6 switches the mode for outputting the image signal from the X-ray detection unit 2 to the second mode. If the image signal output mode is already set to the second mode, the process of step 105 is skipped.

Next, in step 106, the control unit 6 moves the X-ray irradiation range 50. Specifically, the control unit 6 controls the collimator 3 (see FIG. 8) to move the X-ray irradiation range 50. The process of step 106 may be performed after step 102.

Next, in step 107, the control unit 6 controls the X-ray irradiation unit 1 (see FIG. 2), the X-ray detection unit 2, the collimator 3, and the image generation unit 4 (see FIG. 2) to generate an X-ray image 40.

Next, in step 108, the control unit 6 causes the display unit 5 (see FIG. 2) to display the generated X-ray image 40.

Next, in step 109, the control unit 6 determines whether to end the generation of the X-ray image 40. For example, the control unit 6 determines whether to end the generation of the X-ray image 40 based on whether there has been an operation input to end the generation of the X-ray image 40. If the generation of the X-ray image 40 is not to be ended, the process returns to step 101. If the generation of the X-ray image 40 is to be ended, the process terminates.

### Effects of the First Embodiment

With the first embodiment, the following effects can be obtained.

In the first embodiment, as described above, the X-ray imaging apparatus 100 comprises: an X-ray irradiation unit 1 that irradiates X-rays; an X-ray detection unit 2 that has an opposing surface 20 facing the X-ray irradiation unit 1 and outputs an image signal based on the X-rays irradiated by the X-ray irradiation unit 1; a collimator 3 that changes the position of the irradiation range 50 of the X-rays irradiated from the X-ray irradiation unit 1 on the opposing surface 20; an image generation unit 4 that generates an X-ray image 40 based on the image signal output by the X-ray detection unit 2; a display unit 5 that displays the X-ray image 40 generated by the image generation unit 4; and a control unit 6. The X-ray detection unit 2 has a first region 20a, which is a predetermined region on the opposing surface 20, and is capable of switching an output mode between a first mode, in which the time required for outputting the image signal in a predetermined range of the opposing surface 20 is relatively long but the visibility of the X-ray image 40 generated by the image generation unit 4 is relatively high, and a second mode, in which the time required for outputting the image signal in the predetermined range of the opposing surface 20 is relatively small but the visibility of the X-ray image 40 generated by the image generation unit 4 is relatively low. The control unit 6 determines, as a result of the change in the position of the X-ray irradiation range 50 on the opposing surface 20 by the collimator 3, whether it is in a first state where the entire X-ray irradiation range 50 is included in the first region 20a, or a second state where at least a part of the X-ray irradiation range 50 is located within the opposing surface 20 and in a second region 20b outside the first region 20a, and (a) if it is determined to be in the first state, switches the output mode of the X-ray detection unit 2 to the first mode, and (b) if it is determined to be in the second state, switches the output mode of the X-ray detection unit 2 to the second mode.

As a result, when the positional relationship between the first and second regions (20a, 20b) and the X-ray irradiation range 50 is in the first state, the control unit 6 sets the output mode of the X-ray detection unit 2 to the first mode, and when it is in the second state, it sets the output mode to the second mode. This causes the first mode, which generates an X-ray image 40 with relatively high visibility, and the second mode, which generates an X-ray image 40 with relatively low visibility, to be automatically switched. Therefore, the image signal output mode appropriate for the positional relationship between the first and second regions (20a, 20b) and the X-ray irradiation range 50 can be set without the operator having to change the image signal output mode of the X-ray detection unit 2. Consequently, it is possible to suppress complicated operations and temporary interruption of subject observation, which are caused by the operation of changing the output mode of the image signal in the X-ray detection unit 2 accompanying the movement of the X-ray irradiation range 50.

Furthermore, in the first embodiment described above, the following additional effects can be obtained due to the following configuration.

That is, in the first embodiment, as described above, the X-ray image 40 is generated as a moving image, the visibility of the X-ray image 40 is determined by at least one of the frame rate and the resolution, and the control unit 6 is configured to make the visibility of the X-ray image in the second mode relatively low by making at least one of the frame rate and the resolution of the X-ray image 40 generated based on the image signal output in the second mode lower than at least one of the frame rate and the resolution of the X-ray image 40 generated based on the image signal output in the first mode. This allows the visibility of the X-ray image 40 generated based on the image signal output in the second mode to be easily made lower than the visibility of the X-ray image 40 generated based on the image signal output in the first mode by setting the image signal output mode such that at least one of the frame rate or resolution of the X-ray image 40 generated based on the image signal output in the second mode is lowered. As a result, even when the image signal output mode must be changed according to the relative position of the X-ray irradiation range 50 and the first and second regions 20a and 20b, by changing to the second mode, the generation of the X-ray image 40 as a moving image can be seamlessly continued while reducing the visibility of the generated X-ray image 40. For example, if the image signal output mode is set to lower either the frame rate or the resolution of the X-ray image 40 generated based on the image signal output in the second mode, the frame rate or resolution of the X-ray image 40 generated based on the image signal output in the first mode can be maintained at the frame rate or resolution of the X-ray image 40 generated by the image signal output in the first mode. As a result, even when generating the X-ray image 40 based on the image signal output in the second mode, it becomes possible to make either the frame rate or the resolution of the generated X-ray image 40 equal to either the frame rate or the resolution of the X-ray image 40 generated based on the image signal output in the first mode, thereby suppressing an extreme drop in visibility of the generated X-ray image 40 when setting to the second mode.

Furthermore, in the first embodiment, as described above, the apparatus further comprises an input receiving unit 7 that receives an operation input from an operator, and the control unit 6 is configured to allow setting, based on the operation input received via the input receiving unit 7, whether to lower the frame rate of the X-ray image 40 or to lower the resolution of the X-ray image 40 when making the visibility of the X-ray image 40 generated based on the image signal output in the second mode lower than the visibility of the X-ray image 40 generated based on the image signal output in the first mode. When an X-ray image 40 is generated based on the image signal output in the second mode, depending on the surgical procedure, the imaging site, and the operator's preference, there may be cases where one wishes to maintain the frame rate of the generated X-ray image 40 and cases where one wishes to maintain the resolution. Therefore, by configuring the apparatus as described above, it is possible to set whether the frame rate of the X-ray image 40 generated based on the image signal output in the second mode should be lower than the frame rate of the X-ray image 40 generated based on the image signal output in the first mode, or whether the resolution of the X-ray image 40 generated based on the image signal output in the second mode should be lower than the resolution of the X-ray image 40 generated based on the image signal output in the first mode. This allows the operator to set the visibility of the X-ray image 40 generated based on the image signal output in the second mode according to their preference. As a result, the visibility of the X-ray image 40 generated based on the image signal output in the second mode can be set according to the surgical procedure, the imaging site, and the operator's preference, which can improve the operator's convenience (usability).

### Second Embodiment

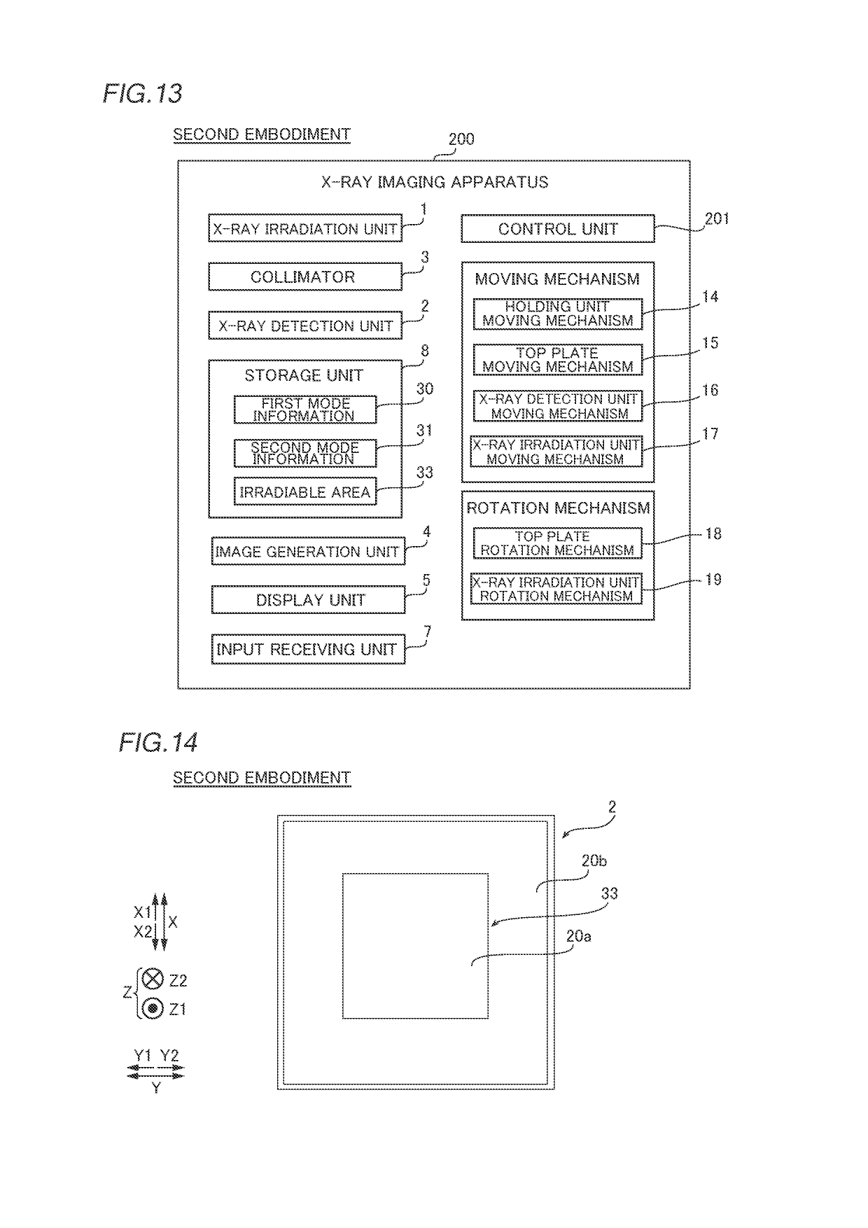

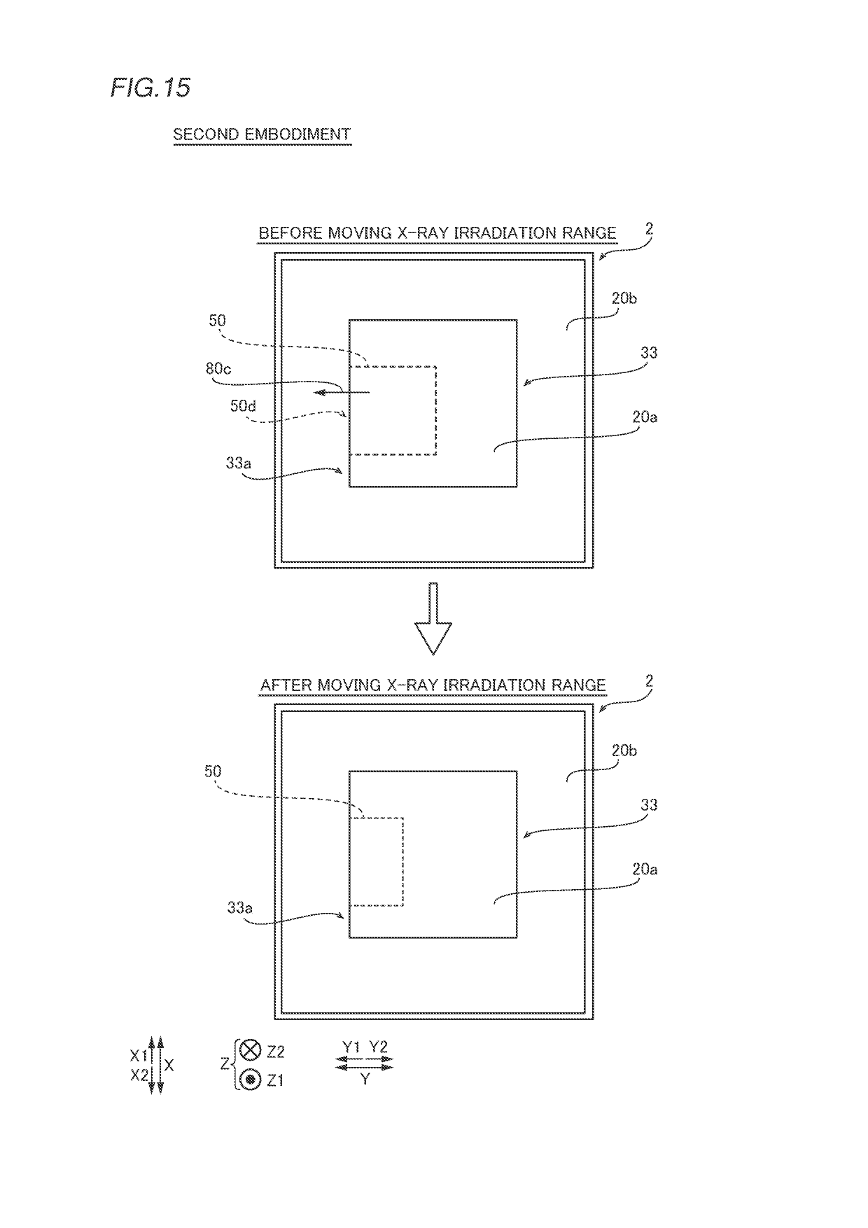

Next, with reference to FIG. 13 to FIG. 18, a second embodiment will be described. In this second embodiment, unlike the first embodiment where the control unit 6 performs control to change the mode for outputting the image signal from the X-ray detection unit 2 from the first mode to the second mode when the X-ray irradiation range 50 moves to a position straddling both the first region 20a and the second region 20b, an example of a configuration will be described in which the control unit 201 deforms the X-ray irradiation range 50 (see FIG. 15) and sets the mode for outputting the image signal from the X-ray detection unit 2 to the first mode, which has relatively higher visibility for the X-ray image 40 compared to the second mode, when the X-ray irradiation range 50 moves to a position straddling both the first region 20a (see FIG. 15) and the second region 20b (see FIG. 15). Note that the same components as in the first embodiment are denoted by the same reference numerals, and their description is omitted.

### Configuration of X-ray Imaging Apparatus

As shown in FIG. 13, an X-ray imaging apparatus 200 includes an X-ray irradiation unit 1, an X-ray detection unit 2, a collimator 3, a control unit 201, an image generation unit 4, a display unit 5, an input receiving unit 7, and a storage unit 8.

In the second embodiment, the control unit 201 is configured to perform control to move the region of interest 51 in the X-ray image 40. The control unit 201 is also configured to perform control to move the X-ray irradiation range 50 based on the movement of the region of interest 51. The control unit 201 is configured to determine, as a result of the change in the position of the X-ray irradiation range 50 on the opposing surface 20 by the collimator 3, whether the apparatus is in a first state where the entire X-ray irradiation range 50 is included in a first region 20a, or a second state where at least a part of the X-ray irradiation range 50 is located within the opposing surface 20 but in a second region 20b outside the first region 20a. The control unit 201 is also configured to, even when it is determined to be in the first state, deform the X-ray irradiation range 50 to maintain the first state and set the output mode of the image signal in the X-ray detection unit 2 to a first mode. (Correction: The original text seems to have a logical error here. It likely means "if it is determined to be in the second state, it deforms the range to maintain the first state". The translation follows this corrected logic based on the overall context of the second embodiment.)

The storage unit 8 is configured to store an irradiable region 33, which will be described later.

### Setting of Irradiable Region

In the second embodiment, as shown in FIG. 14, the control unit 201 is configured to perform control to set an irradiable region 33 for X-rays irradiated from the X-ray irradiation unit 1 on the opposing surface 20. The control unit 201 sets the irradiable region 33 based on an operator's operation input. In the example shown in FIG. 14, the irradiable region 33 is set to the entire first region 20a. The control unit 201 stores the set irradiable region 33 in the storage unit 8 (see FIG. 13). Specifically, the control unit 201 stores the position information of the pixels 2a set as the irradiable region 33 among the pixels of the opposing surface 20 of the X-ray detection unit 2 in the storage unit 8.

### Deformation of X-ray Irradiation Range

Next, with reference to FIG. 15, a configuration in which the control unit 201 (see FIG. 13) of the second embodiment performs control to deform the X-ray irradiation range 50 will be described. FIG. 15 shows an example of moving the X-ray irradiation range 50 in the Y1 direction, as indicated by arrow 80c, from a position where the Y1-direction-side end 50d of the X-ray irradiation range 50 is at the same position as the Y1-direction-side end 33a of the irradiable region 33. The control unit 201 of the second embodiment performs control to deform the shape of the X-ray irradiation range 50 to match the end 33a of the irradiable region 33 when moving the X-ray irradiation range 50 such that its end 50d is positioned outside the end 33a of the irradiable region 33. In the example shown in FIG. 15, the shape of the X-ray irradiation range 50 before movement is a square, but the X-ray irradiation range 50 after movement is deformed to have a rectangular shape.

### Display of Frame Line Corresponding to X-ray Irradiation Range

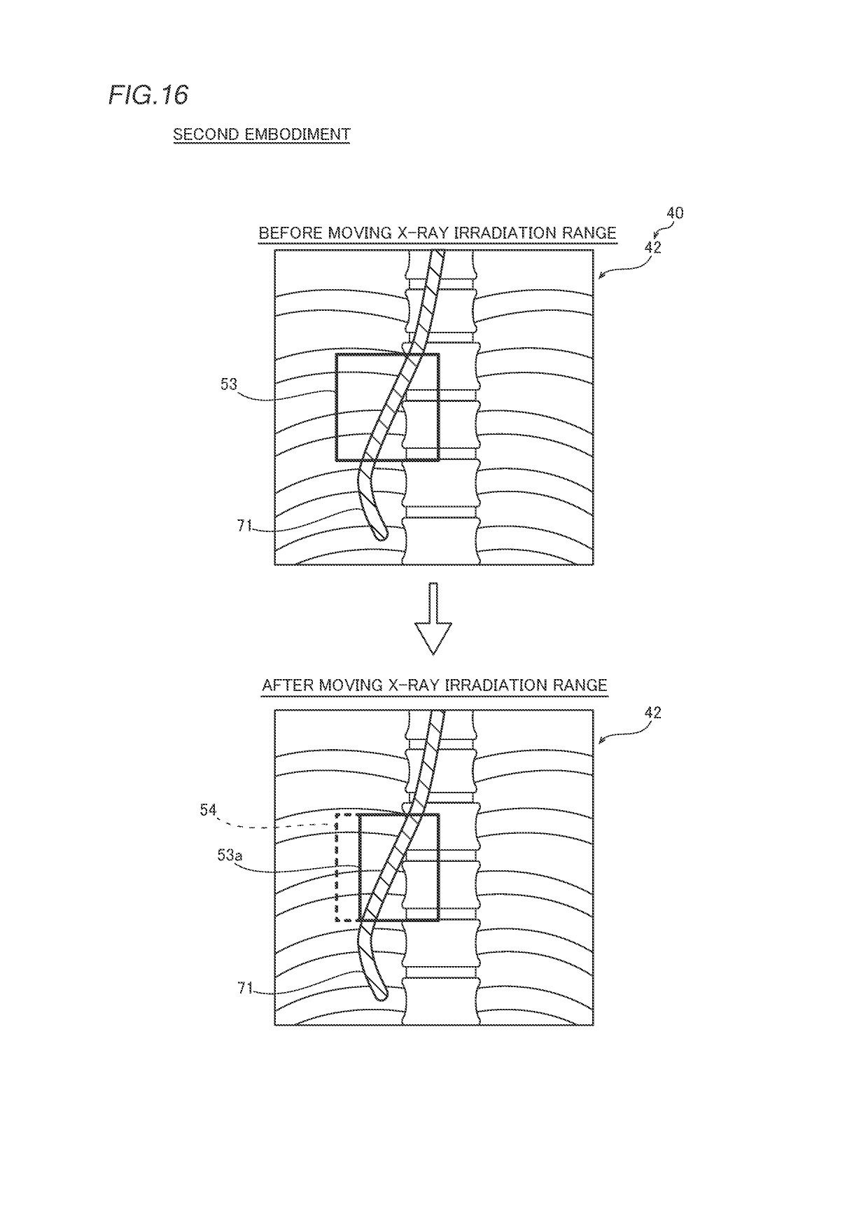

Next, with reference to FIG. 16, a configuration will be described in which the control unit 201 (see FIG. 13) of the second embodiment displays a frame line 53 corresponding to the X-ray irradiation range 50 (see FIG. 15) on the X-ray image 40.

As shown in FIG. 16, the control unit 201 is configured to perform control to superimpose and display a frame line 53, which indicates the X-ray irradiation range 50, on an overall X-ray image 42, which is an X-ray image generated based on the image signal output from the entire area of the opposing surface 20. In the second embodiment, the control unit 201 deforms the X-ray irradiation range 50 when it is moved such that the end 50d (see FIG. 15) of the X-ray irradiation range 50 is positioned outside the end 33a (see FIG. 15) of the irradiable region 33 (see FIG. 15). In this case, if a frame line that does not correspond to the deformation of the X-ray irradiation range 50 is displayed, a frame line 54 including a region that is not actually irradiated with X-rays will be displayed. This would result in the capturing of an X-ray image 40 where a region different from what the operator expects is imaged.

Therefore, in the second embodiment, the control unit 201 is configured to perform control to deform and display the frame line 53 to match the shape of the deformed X-ray irradiation range 50 when the X-ray irradiation range 50 is moved such that its end 50d is positioned outside the end 33a of the irradiable region 33. In the example shown in FIG. 15, since the X-ray irradiation range 50 is deformed into a rectangle, the frame line 53 is deformed into a rectangle in the example shown in FIG. 16. The frame line 53 is a line indicating the region corresponding to the range actually irradiated with X-rays, and may indicate a region different from the region of interest 51 depending on the deformation of the X-ray irradiation range 50. The positional relationship of the frame line 53 with respect to the overall X-ray image 42 is the same as the positional relationship of the X-ray irradiation range 50 with respect to the opposing surface 20 (see FIG. 14) of the X-ray detection unit 2 (see FIG. 14). The positional relationship of the X-ray irradiation range 50 with respect to the opposing surface 20 of the X-ray detection unit 2 can be obtained based on position information that can be acquired from a drive unit that drives the X-ray detection unit 2, a drive unit that drives the collimator 3 (see FIG. 13), and the like.

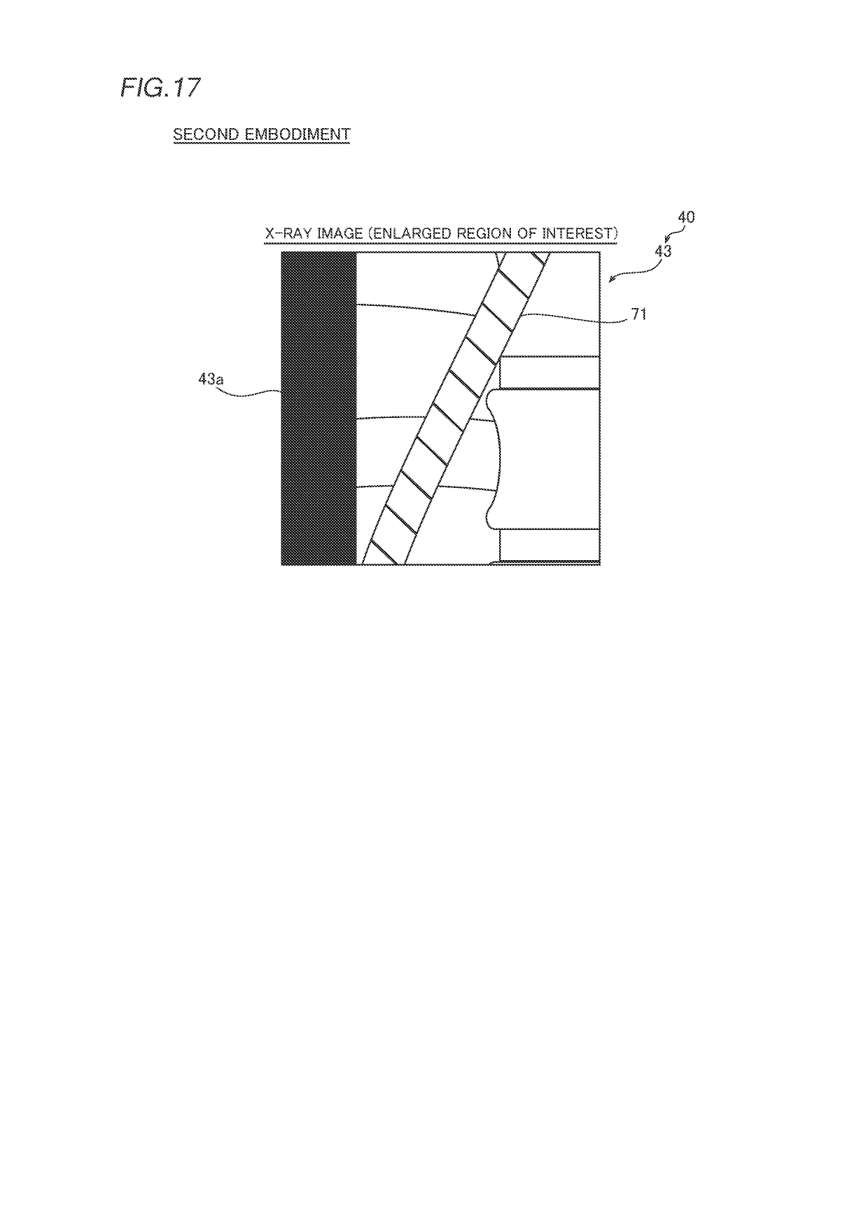

In the second embodiment, when the X-ray irradiation range 50 is moved such that its end 50d is positioned outside the end 33a of the irradiable region 33, the X-ray irradiation range 50 is deformed. Therefore, as shown in FIG. 17, the image generation unit 4 (see FIG. 13) generates an X-ray image 43 that displays a portion corresponding to the area not included in the deformed X-ray irradiation range 50 within the region of interest 51 of the X-ray image 40 as a padding region 43a. Then, the control unit 201 causes the display unit 5 to display the X-ray image 43. Since the padding region 43a is a region that is not irradiated with X-rays, no image signal is output from the corresponding pixels 2a. Therefore, the padding region 43a is treated as having a pixel value of "0" and is displayed in black or white. The X-ray image 43 shown in FIG. 17 is an example where the padding region 43a is displayed in black.

### X-ray Irradiation Range Adjustment Process

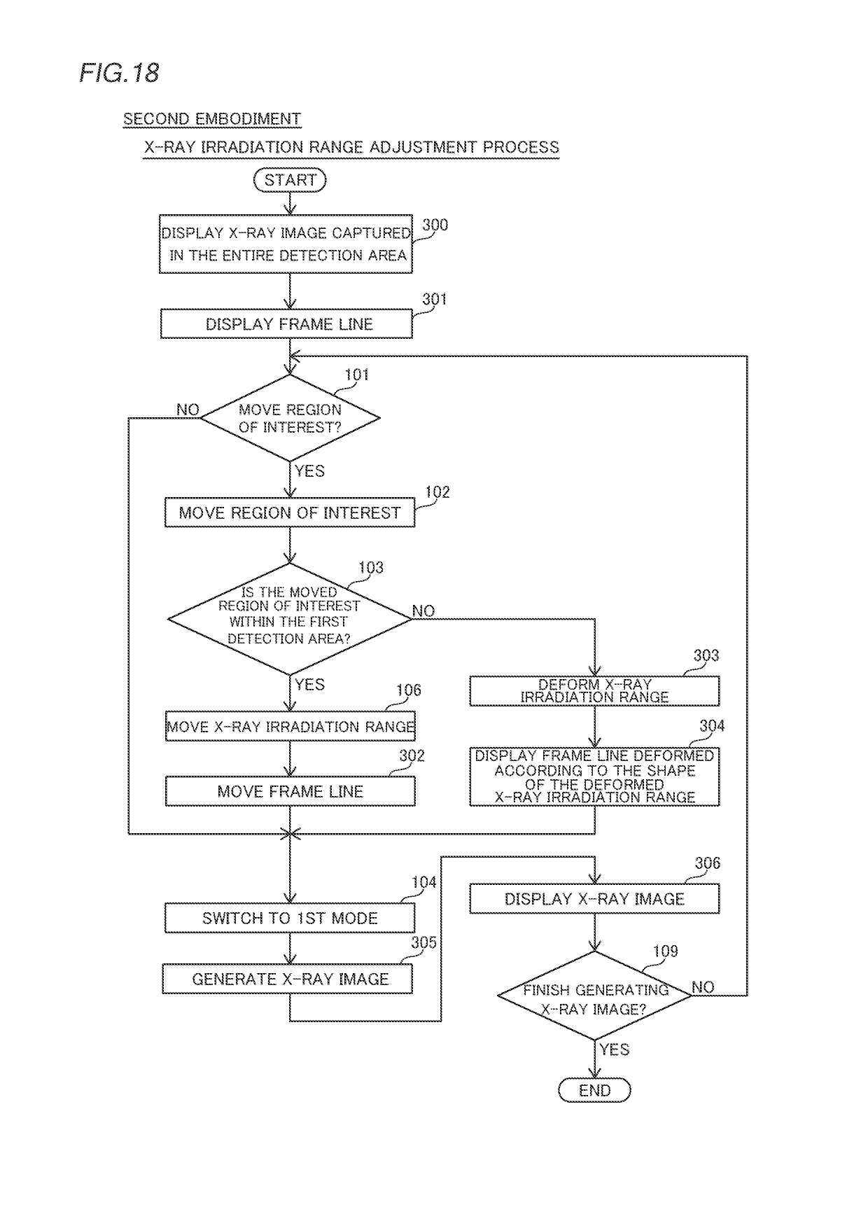

Next, with reference to FIG. 18, a process in which the control unit 201 (see FIG. 13) adjusts the X-ray irradiation range 50 (see FIG. 15) when generating the X-ray image 40 (see FIG. 16) will be described.

In step 300, the image generation unit 4 generates an overall X-ray image 42 (see FIG. 16), which is an X-ray image 40 generated based on the X-rays detected in the entire area of the opposing surface 20. Then, the control unit 201 causes the display unit 5 (see FIG. 13) to display the generated overall X-ray image 42.

Next, the control unit 201 superimposes and displays a frame line 53 (see FIG. 16), which indicates the X-ray irradiation range 50 (see FIG. 15), on the overall X-ray image 42.

Then, the process proceeds from step 101 to step 103. In step 103, if the moved region of interest 51 is located within the first region 20a, the process proceeds to step 106, and then to step 302. If the moved region of interest 51 is not located within the first region 20a, the process proceeds to step 303.

When the process proceeds from step 103 through step 106 to step 302, in step 302, the control unit 201 moves the frame line 53 (see FIG. 17) to match the movement of the X-ray irradiation range 50. At this time, the control unit 201 does not deform the X-ray irradiation range 50 and the frame line 53. Thereafter, the process proceeds through step 104 to step 305.

When the process proceeds from step 103 to step 303, in step 303, the control unit 201 deforms the X-ray irradiation range 50. Specifically, the control unit 201 deforms the shape of the X-ray irradiation range 50 to match the end 33a (see FIG. 15) of the irradiable region 33 (see FIG. 15).

Next, in step 304, the control unit 201 deforms the frame line 53. Specifically, the control unit 201 deforms the frame line 53 to match the shape of the deformed X-ray irradiation range 50.

Thereafter, the process proceeds through step 104 to step 305. In step 305, the image generation unit 4 generates an X-ray image 40. When the process proceeds to step 305 via steps 103, 106, 302, and 104, the image generation unit 4 generates the X-ray image 40 in the same manner as in the first embodiment. When the process proceeds to step 305 via steps 103, 303, 304, and 104, the image generation unit 4 generates the X-ray image 43 shown in FIG. 17.

Next, in step 306, the control unit 201 displays the X-ray image 40 or the X-ray image 43 on the display unit 5.

Then, in step 109, a determination is made as to whether to end the generation of the X-ray image 40 or X-ray image 43. If the generation is not to be ended, the process returns to step 101. If the generation is to be ended, the process terminates.

Other configurations of the second embodiment are the same as those of the first embodiment.

### Effects of the Second Embodiment

With the second embodiment, the following effects can be obtained.

In the second embodiment, as described above, the X-ray imaging apparatus 200 comprises: an X-ray irradiation unit 1 that irradiates X-rays; an X-ray detection unit 2 that has an opposing surface 20 facing the X-ray irradiation unit 1 and outputs an image signal based on the X-rays irradiated by the X-ray irradiation unit 1; a collimator 3 that changes the position of the irradiation range 50 of the X-rays irradiated from the X-ray irradiation unit 1 on the opposing surface 20; an image generation unit 4 that generates an X-ray image 40 based on the image signal output by the X-ray detection unit 2; a display unit 5 that displays the X-ray image 40 generated by the image generation unit 4; and a control unit 201. The X-ray detection unit 2 has a first region 20a, which is a predetermined region on the opposing surface 20, and is capable of switching an output mode between a first mode, in which the time required for outputting the image signal in a predetermined range of the opposing surface 20 is relatively long but the visibility of the X-ray image 40 generated by the image generation unit 4 is relatively high, and a second mode, in which the time required for outputting the image signal in the predetermined range of the opposing surface 20 is relatively small but the visibility of the X-ray image 40 generated by the image generation unit 4 is relatively low. The control unit 201 determines, as a result of the change in the position of the X-ray irradiation range 50 on the opposing surface 20 by the collimator 3, whether it is in a first state where the entire X-ray irradiation range 50 is included in the first region 20a, or a second state where at least a part of the X-ray irradiation range 50 is located within the opposing surface 20 and in a second region 20b outside the first region 20a, and if it is determined to be in the second state, deforms the X-ray irradiation range 50 to maintain the first state and sets the output mode of the image signal in the X-ray detection unit 2 to the first mode.

As a result, the mode for outputting the image signal from the X-ray detection unit 2 is not changed from the first mode, which has relatively high visibility for the X-ray image 40, due to the movement of the X-ray irradiation range 50. This suppresses complicated operations and temporary interruption of subject observation caused by changing the mode of outputting the image signal to the second mode accompanying the movement of the X-ray irradiation range 50. Some operators may not need the X-ray image 40 of parts of the subject other than the part they wish to observe. Therefore, some operators may prefer that when the X-ray irradiation range 50 moves to a position straddling the first region 20a and the second region 20b, the mode is set to the first mode, which generates an X-ray image 40 with relatively high visibility, rather than being changed to the second mode with relatively low visibility. In the X-ray imaging apparatus 200 of the second embodiment, even when the control unit 201 determines that the apparatus is in the second state where at least a part of the X-ray irradiation range 50 is included in the second region 20b as a result of the change in the position of the X-ray irradiation range 50 on the opposing surface 20 by the collimator 3, it deforms the X-ray irradiation range 50 to maintain the first state and sets the output mode of the image signal in the X-ray detection unit 2 to the first mode. As a result, even when it is determined that the apparatus is in the second state where at least a part of the X-ray irradiation range 50 is included in the second region 20b, the first mode, which outputs only the image signals of the pixels 2a located within the first region 20a, can be maintained. Consequently, it is possible to provide an X-ray imaging apparatus 200 that can meet the needs of operators who wish for the mode to be set to one that outputs only the image signals of the pixels 2a located within the first region 20a, without outputting the image signals from the region of the X-ray irradiation range 50 located outside the first region 20a.

Furthermore, in the second embodiment described above, the following additional effects can be obtained due to the following configuration.

That is, in the second embodiment, as described above, the control unit 201 performs control to set an irradiable region 33 for the X-rays irradiated from the X-ray irradiation unit 1 on the opposing surface 20, and control to deform the shape of the X-ray irradiation range 50 to match the end 33a of the irradiable region 33 when the X-ray irradiation range 50 is moved such that its end 50d is positioned outside the end 33a of the irradiable region 33. This can suppress the irradiation of unnecessary X-rays, which are not used for imaging, outside the irradiable region 33. As a result, unnecessary radiation exposure can be suppressed.

Furthermore, in the second embodiment, as described above, the control unit 201 performs control to superimpose and display a frame line 53, which indicates the X-ray irradiation range 50, on an overall X-ray image 42, which is an X-ray image 40 generated based on the X-rays detected in the entire area of the opposing surface 20, and control to deform and display the frame line 53 to match the shape of the deformed X-ray irradiation range 50 when the X-ray irradiation range 50 is moved such that its end 50d is positioned outside the end 33a of the irradiable region 33. As a result, the frame line 53 is displayed in a deformed state matching the shape of the deformed X-ray irradiation range 50, so the operator can easily grasp the range that is actually irradiated with X-rays by checking the deformed frame line 53. Consequently, compared to a configuration that does not deform the frame line 53 to match the shape of the deformed X-ray irradiation range 50, it becomes possible for the operator to easily grasp the range that is actually irradiated with X-rays, which can improve the operator's convenience (usability).

Other effects of the second embodiment are the same as those of the first embodiment.

### Modifications

It should be understood that the embodiments disclosed herein are illustrative and not restrictive in all respects. The scope of the present invention is indicated by the claims rather than by the description of the embodiments, and all changes (modifications) that come within the meaning and range of equivalency of the claims are intended to be embraced therein.

For example, the frame rate and resolution (binning size) of the X-ray image 40 generated based on the image signal output in the first mode, and the frame rate and resolution (binning size) of the X-ray image 40 generated based on the image signal output in the second mode in the first and second embodiments are merely examples. The conditions for reading out the charge may be set to other fps and binning sizes, as long as the visibility of the X-ray image generated based on the image signal output in the first mode is improved compared to the visibility of the X-ray image generated based on the image signal output in the second mode.

In the first embodiment, as shown in the second embodiment, the configuration may be such that an overall X-ray image is displayed on the display unit, and a frame line is superimposed and displayed on the displayed overall X-ray image.

In the first embodiment, an example was shown where the control unit 6 is configured to allow the operator to set, based on an operation input via the input receiving unit 7, whether to lower the frame rate or the resolution of the X-ray image 40 when making the visibility of the X-ray image 40 generated based on the image signal output in the second mode lower than the visibility of the X-ray image 40 generated based on the image signal output in the first mode. However, the present invention is not limited to this. For example, the frame rate in the second mode or the resolution in the second mode may be preset and not be changeable by the operator's operation input. However, if the frame rate in the second mode or the resolution in the second mode cannot be changed by the operator's operation input, the visibility of the X-ray image generated based on the image signal output in the second mode cannot be set according to the operator's preference, which reduces the operator's convenience (usability). Therefore, it is preferable that the control unit is configured to allow setting whether to lower the frame rate or the resolution when making the visibility of the X-ray image generated based on the image signal output in the second mode lower than the visibility of the X-ray image generated based on the image signal output in the first mode.

In the first embodiment, an example was shown of a configuration in which the image signal output mode in the second mode results in an image signal output mode where either the frame rate or the resolution is lower than that of the X-ray image 40 generated based on the image signal output in the first mode. However, the present invention is not limited to this. For example, the control unit may be configured to set the image signal output mode in the second mode to an image signal output mode that generates an X-ray image with both a lower frame rate and lower resolution than the X-ray image generated based on the image signal output in the first mode.

In the first and second embodiments, an example was shown where the X-ray imaging apparatus 100 (200) is configured as a so-called fluoroscopy table equipped with a top plate 9, but the present invention is not limited to this. For example, the present invention can be applied to devices other than fluoroscopy tables, as long as they are X-ray imaging apparatuses that capture X-ray images as moving images.

In the first and second embodiments, an example was shown of a configuration in which the collimator 3 (X-ray irradiation range adjustment unit) has first to fourth shielding blades 131-134 and fifth to eighth shielding blades 141-144, but the present invention is not limited to this. The collimator (X-ray irradiation range adjustment unit) may have any configuration as long as it can change the X-ray irradiation range.

In the first and second embodiments, an example was shown of a configuration in which the X-ray detection unit 2 has a first region 20a and a second region 20b on the opposing surface 20, but the present invention is not limited to this. The X-ray detection unit may have three or more regions on the opposing surface.

Although the process in which the control unit 6 of the first embodiment changes the mode for outputting the image signal from the X-ray detection unit 2, and the process in which the control unit 201 of the second embodiment adjusts the X-ray irradiation range 50 have been described using flow-driven flowcharts that process in sequence along the process flow, the present invention is not limited to this. In the present invention, the processing performed by the control unit may be performed by event-driven processing that executes processing on an event basis. In this case, it may be performed in a completely event-driven manner, or by a combination of event-driven and flow-driven processing.

### Aspects

It will be understood by those skilled in the art that the exemplary embodiments described above are specific examples of the following aspects.

### Item 1

An X-ray imaging apparatus, comprising: an X-ray irradiation unit that irradiates X-rays; an X-ray detection unit that has an opposing surface facing the X-ray irradiation unit and outputs an image signal based on the X-rays irradiated by the X-ray irradiation unit; an irradiation range changing unit that changes a position of an irradiation range of the X-rays irradiated from the X-ray irradiation unit on the opposing surface; an image generation unit that generates an X-ray image based on the image signal output by the X-ray detection unit; a display unit that displays the X-ray image generated by the image generation unit; and a control unit, wherein the X-ray detection unit: has a first region, which is a predetermined region on the opposing surface, and is capable of switching an output mode between a first mode, in which a time required for outputting the image signal in a predetermined range of the opposing surface is relatively long but a visibility of the X-ray image generated by the image generation unit is relatively high, and a second mode, in which the time required for outputting the image signal in the predetermined range of the opposing surface is relatively small but the visibility of the X-ray image generated by the image generation unit is relatively low, and the control unit: determines, as a result of the change in the position of the X-ray irradiation range on the opposing surface by the irradiation range changing unit, whether the apparatus is in a first state where an entire X-ray irradiation range is included in the first region, or a second state where at least a part of the X-ray irradiation range is located within a second region outside the first region in the opposing surface, and (a) if it is determined to be in the first state, switches the output mode of the X-ray detection unit to the first mode, and (b) if it is determined to be in the second state, switches the output mode of the X-ray detection unit to the second mode.

### Item 2

The X-ray imaging apparatus according to item 1, wherein the X-ray image is generated as a moving image, the visibility of the X-ray image is determined by at least one of a frame rate and a resolution, and the control unit is configured to make the visibility of the X-ray image in the second mode relatively low by making at least one of the frame rate and the resolution of the X-ray image generated based on the image signal output in the second mode lower than at least one of the frame rate and the resolution of the X-ray image generated based on the image signal output in the first mode.

### Item 3

The X-ray imaging apparatus according to item 1 or 2, further comprising an input receiving unit that receives an operation input from an operator, wherein the control unit is configured to allow setting, based on the operation input received via the input receiving unit, whether to lower the frame rate of the X-ray image or to lower the resolution of the X-ray image when making the visibility of the X-ray image generated based on the image signal output in the second mode lower than the visibility of the X-ray image generated based on the image signal output in the first mode.

### Item 4

An X-ray imaging apparatus, comprising: an X-ray irradiation unit that irradiates X-rays; an X-ray detection unit that has an opposing surface facing the X-ray irradiation unit and outputs an image signal based on the X-rays irradiated by the X-ray irradiation unit; an irradiation range changing unit that changes a position of an irradiation range of the X-rays irradiated from the X-ray irradiation unit on the opposing surface; an image generation unit that generates the X-ray image based on the image signal output by the X-ray detection unit; a display unit that displays the X-ray image generated by the image generation unit; and a control unit, wherein the X-ray detection unit: has a first region, which is a predetermined region on the opposing surface, and is capable of switching an output mode between a first mode, in which a time required for outputting the image signal in a predetermined range of the opposing surface is relatively long but a visibility of the X-ray image generated by the image generation unit is relatively high, and a second mode, in which the time required for outputting the image signal in the predetermined range of the opposing surface is relatively small but the visibility of the X-ray image generated by the image generation unit is relatively low, and the control unit: determines, as a result of the change in the position of the X-ray irradiation range on the opposing surface by the irradiation range changing unit, whether the apparatus is in a first state where an entire X-ray irradiation range is included in the first region, or a second state where at least a part of the X-ray irradiation range is located within a second region outside the first region in the opposing surface, and if it is determined to be in the second state, deforms the X-ray irradiation range to maintain the first state and sets the output mode of the image signal in the X-ray detection unit to the first mode.

### Item 5

The X-ray imaging apparatus according to item 4, wherein the control unit performs: control to set an irradiable region for the X-rays irradiated from the X-ray irradiation unit on the opposing surface, and control to deform a shape of the X-ray irradiation range to match an end of the irradiable region when moving the X-ray irradiation range such that an end of the X-ray irradiation range is positioned outside the end of the irradiable region.

### Item 6

The X-ray imaging apparatus according to item 5, wherein the control unit performs: control to superimpose and display a frame line, which indicates the X-ray irradiation range, on an overall X-ray image, which is the X-ray image generated based on the X-rays detected in an entire area of the opposing surface, and control to deform and display the frame line to match the shape of the deformed X-ray irradiation range when moving the X-ray irradiation range such that the end of the X-ray irradiation range is positioned outside the end of the irradiable region.

### REFERENCE SIGNS LIST

1 X-ray irradiation unit

2 X-ray detection unit

3 Collimator (irradiation range changing unit)

4 Image generation unit

5 Display unit

6, 201 Control unit

7 Input receiving unit

20 Opposing surface (opposing surface of X-ray detection unit)

20a First region (predetermined region on opposing surface)

20b Second region (region within the opposing surface and outside the first region)

32 Threshold

33 Irradiable region

33a End of irradiable region

40, 41, 43 X-ray image

42 Overall X-ray image

50, 50a, 50b X-ray irradiation range

51 Region of interest

53 Frame line (frame line indicating X-ray irradiation range)

100, 200 X-ray imaging apparatus

1. An X-ray imaging apparatus, comprising:
an X-ray irradiation unit that irradiates X-rays;
an X-ray detection unit that has an opposing surface facing the X-ray irradiation unit and outputs an image signal based on the X-rays irradiated by the X-ray irradiation unit;
an irradiation range changing unit that changes a position of an irradiation range of the X-rays irradiated from the X-ray irradiation unit on the opposing surface;
an image generation unit that generates an X-ray image based on the image signal output by the X-ray detection unit;
a display unit that displays the X-ray image generated by the image generation unit; and
a control unit,
wherein the X-ray detection unit:
has a first region, which is a predetermined region on the opposing surface, and
is capable of switching an output mode between a first mode, in which a time required for outputting the image signal in a predetermined range of the opposing surface is relatively long but a visibility of the X-ray image generated by the image generation unit is relatively high, and a second mode, in which the time required for outputting the image signal in the predetermined range of the opposing surface is relatively small but the visibility of the X-ray image generated by the image generation unit is relatively low, and
wherein the control unit:
determines, as a result of the change in the position of the X-ray irradiation range on the opposing surface by the irradiation range changing unit, whether the apparatus is in a first state where an entire X-ray irradiation range is included in the first region, or a second state where at least a part of the X-ray irradiation range is located within a second region outside the first region in the opposing surface, and
(a) if it is determined to be in the first state, switches the output mode of the X-ray detection unit to the first mode, and
(b) if it is determined to be in the second state, switches the output mode of the X-ray detection unit to the second mode.

2. The X-ray imaging apparatus according to claim 1, wherein
the X-ray image is generated as a moving image,
the visibility of the X-ray image is determined by at least one of a frame rate and a resolution, and
the control unit is configured such that the X-ray image generated based on the image signal output in the second mode has its visibility made relatively low by making at least one of the frame rate and the resolution lower than at least one of the frame rate and the resolution of the X-ray image generated based on the image signal output in the first mode.

3. The X-ray imaging apparatus according to claim 2, further comprising an input receiving unit that receives an operation input from an operator,
wherein the control unit is configured to allow setting, based on the operation input received via the input receiving unit, whether to lower the frame rate of the X-ray image or to lower the resolution of the X-ray image when making the visibility of the X-ray image generated based on the image signal output in the second mode lower than the visibility of the X-ray image generated based on the image signal output in the first mode.

4. An X-ray imaging apparatus, comprising:
an X-ray irradiation unit that irradiates X-rays;
an X-ray detection unit that has an opposing surface facing the X-ray irradiation unit and outputs an image signal based on the X-rays irradiated by the X-ray irradiation unit;
an irradiation range changing unit that changes a position of an irradiation range of the X-rays irradiated from the X-ray irradiation unit on the opposing surface;
an image generation unit that generates an X-ray image based on the image signal output by the X-ray detection unit;
a display unit that displays the X-ray image generated by the image generation unit; and
a control unit,
wherein the X-ray detection unit:
has a first region, which is a predetermined region on the opposing surface, and
is capable of switching an output mode between a first mode, in which a time required for outputting the image signal in a predetermined range of the opposing surface is relatively long but a visibility of the X-ray image generated by the image generation unit is relatively high, and a second mode, in which the time required for outputting the image signal in the predetermined range of the opposing surface is relatively small but the visibility of the X-ray image generated by the image generation unit is relatively low, and
wherein the control unit:
determines, as a result of the change in the position of the X-ray irradiation range on the opposing surface by the irradiation range changing unit, whether the apparatus is in a first state where an entire X-ray irradiation range is included in the first region, or a second state where at least a part of the X-ray irradiation range is located within a second region outside the first region in the opposing surface, and
if it is determined to be in the second state, deforms the X-ray irradiation range to maintain the first state and sets the output mode of the image signal in the X-ray detection unit to the first mode.

5. The X-ray imaging apparatus according to claim 4, wherein the control unit performs:
control to set an irradiable region for the X-rays irradiated from the X-ray irradiation unit on the opposing surface, and
control to deform a shape of the X-ray irradiation range to match an end of the irradiable region when moving the X-ray irradiation range such that an end of the X-ray irradiation range is positioned outside the end of the irradiable region.

6. The X-ray imaging apparatus according to claim 5, wherein the control unit performs:
control to superimpose and display a frame line, which indicates the X-ray irradiation range, on an overall X-ray image, which is the X-ray image generated based on the X-rays detected in an entire area of the opposing surface, and
control to deform and display the frame line to match the shape of the deformed X-ray irradiation range when moving the X-ray irradiation range such that the end of the X-ray irradiation range is positioned outside the end of the irradiable region.
